(19) United States
(12) Patent Application Publication
(10) Pub. No.: US 2017/0112773 A1
(43) Pub. Date: Apr. 27, 2017

(54) PLASMA MEMBRANE VESICLES COMPRISING FUNCTIONAL TRANSMEMBRANE PROTEINS

(71) Applicant: Board of Regents, the University of Texas System, Austin, TX (US)

(72) Inventors: Jeanne STACHOWIAK, Austin, TX (US); Hugh SMYTH, Austin, TX (US); Avinash Kaur GADOK, Austin, TX (US); Silvia FERRATI, Austin, TX (US); Chi ZHAO, Austin, TX (US)

(21) Appl. No.: 15/333,137

(22) Filed: Oct. 24, 2016

Related U.S. Application Data
(60) Provisional application No. 62/245,665, filed on Oct. 23, 2015.

(51) Int. Cl. A61K 9/50; A61K 31/704; A61K 31/713

(52) U.S. Cl. CPC A61K 9/5052; A61K 31/713; A61K 9/5015; A61K 31/704; A61K 47/48584

(57) ABSTRACT

Provided herein are methods and compositions for treating a subject in need thereof comprising administering an effective amount of vesicles with functional transmembrane proteins embedded in the plasma membrane. Also provided herein are methods of restoring gap junctional communication comprising the administration of an effective amount of vesicles.

The present application claims the priority benefit of U.S. provisional application No. 62/245,665, filed Oct. 23, 2015, the entire contents of which are incorporated herein by reference.

This invention was made with government support under Grant No. DMR1352487 awarded by the National Science Foundation. The government has certain rights in the invention.

### INCORPORATION OF SEQUENCE LISTING

The sequence listing that is contained in the file named “UTSBP1075US_ST25.txt”, which is 1 KB (as measured in Microsoft Windows®) and was created on Oct. 24, 2016, is filed herewith by electronic submission and is incorporated by reference herein.

### BACKGROUND OF THE INVENTION

1. Field of the Invention

The present invention relates generally to the field of medicine and biology. More particularly, it concerns methods and compositions of delivering transmembrane proteins.

2. Description of Related Art

Transmembrane proteins comprise a major group of proteins that perform a wide range of functions and act to translate extracellular signals to intracellular responses. They include G-protein coupled receptors, transporters and metabolic enzymes. Many human diseases are linked to mutations in transmembrane proteins, aberrant localization at different intracellular loci and changes in cellular physiology. For example, mutated or dysregulated transmembrane proteins include gap junction proteins such as Connexin 43 and the cystic fibrosis transmembrane regulator (CFTR). As a result of dysregulated gap junction proteins, many cancers have disrupted gap junctional communication. Gap junctions remain reduced during tumor progression and altered in metastasis. Thus, methods for restoring transmembrane protein function as well as gap junctional communication are needed for the development of therapeutics.

Delivering drugs and reagents across the cell's plasma membrane barrier remains a formidable challenge, despite its fundamental importance to diverse fields including biotechnology, cell biology, and pharmaceutics. Specifically, the difficulty of circumventing the plasma membrane has required most drugs and reagents to be membrane soluble, greatly limiting their design and application. However, cells transport a range of water soluble small molecules, including metabolites, second messengers, drugs, peptides and siRNA, from the cytoplasm of one cell to the next using gap junctions. Gap junctions are networks of transmembrane protein channels that physically connect the cytoplasms of adjacent cells. Thus, a delivery approach that harnesses the gap junction network has the potential to release molecular cargoes directly into the cytoplasm.

### SUMMARY OF THE INVENTION

Embodiments of the present disclosure provide methods and compositions for the treatment of conditions with mutated or dysfunctional transmembrane proteins comprising the administration of recombinant transmembrane proteins. In one embodiment, there is provided a vesicle comprising a phospholipid membrane wherein the phospholipid membrane comprises a recombinant transmembrane protein. In a further embodiment, there is provided a vesicle comprising a phospholipid membrane wherein the phospholipid membrane comprises a recombinant transmembrane protein and a fusogenic peptide, wherein said protein and peptide are embedded in said membrane. In some aspects, the vesicle has a diameter of less than about 20 μm. In other aspects, the vesicle has a diameter of less than about 10 μm. In certain aspects, the fusogenic peptide comprises trans-activating transcriptional activator (TAT) or TAT-HA2. In some aspects, the vesicles have a diameter of about 30 nm to about 100 μm, such as 1 μm to 100 μm, such as 30 nm to 150 nm.

In certain aspects, the recombinant transmembrane protein is a transporter, receptor, channel, cell adhesion protein, or enzyme. In some aspects, the recombinant transmembrane protein is a connexin, cystic fibrosis transmembrane conductance regulator (CFTR), thyrotropin receptor, myelin protein zero, melacortin 4, myelin proteolipid protein, low-density lipoprotein receptor, or ABC transporter. For example, the recombinant transmembrane protein is Connexin 43 or CFTR. In certain aspects, about 100,000 to about 500,000 recombinant transmembrane proteins are embedded in the phospholipid membrane.

In some aspects, the vesicle further comprises a small molecule, peptide, nucleic acid molecule, or RNA. In certain aspects, the vesicle further comprises a chemotherapeutic drug. For example, the chemotherapeutic drug is doxorubicin, etoposide, paclitaxel, or gemcitabine.

In certain aspects, the vesicle further comprises a targeting molecule. In some aspects, the targeting molecule comprises an antibody or fragment thereof, a polypeptide, a dendrimer, an aptamer, an oligomer or a small molecule. In particular aspects, the targeting molecule has an affinity for a receptor expressed in cancer cells. For example, the targeting molecule binds to human epidermal growth factor receptor (EGFR), vascular endothelial growth factor receptor, folic acid receptor, melanocyte stimulating hormone receptor, integrin avb3, integrin avb5, transferrin receptor, interleukin receptors, lectins, insulin-like growth factor receptor, hepatocyte growth factor receptor or basic fibroblast growth factor receptor. In some aspects, the antibody fragment is an EGFR single-domain antibody fragment.

In further aspects, there is provided a method of producing the vesicle provided herein comprising a phospholipid membrane wherein the phospholipid membrane comprises a recombinant transmembrane protein and a fusogenic peptide, wherein said protein and peptide are embedded in said membrane, comprising (a) providing a donor cell, wherein the donor cell is genetically engineered to express the recombinant transmembrane protein, (b) contacting the donor cell with a blebbing buffer, under conditions effective to induce donor cell blebbing, and (c) harvesting the vesicle from the blebbing buffer. In some aspects, the blebbing buffer comprises a sulfhydryl blocking agent and a reducing agent. For example, the sulfhydryl blocking agent is paraformaldehyde. In another embodiment, the method for producing the vesicle provided herein comprises (a) providing a donor cell, wherein the donor cell is genetically engineered to express the recombinant transmembrane protein; (b) contacting the donor cell with a polymer, under conditions effective to induce precipitation of vesicles (e.g., exosomes); and (c) isolating the precipitated vesicles. In some aspects, the polymer is polyethylene glycol, dextran, dextran sulfate, dextran acetate, polyvinyl alcohol, polyvinyl acetate, or polyvinyl sulfate.

In certain aspects, the donor cell comprises an expression construct encoding the recombinant transmembrane protein. In some aspects, the method of producing a vesicle further comprises washing the donor cell prior to contacting the cells with the blebbing buffer or polymer. In certain aspects, the donor cell is a mammalian cell. In some aspects, the donor cell is a human cell.

In further aspects, there is provided a method of producing a vesicle comprising a phospholipid membrane wherein the phospholipid membrane comprises a recombinant transmembrane protein and a fusogenic peptide, wherein said protein and peptide are embedded in said membrane, comprising (a) mixing phospholipids in an organic solvent, (b) adding the recombinant transmembrane protein, and (c) isolating vesicles with the recombinant transmembrane protein.

In another embodiment, there is provided a method of treating a disease or disorder in a subject comprising administering a therapeutically effective amount of the vesicle provided herein comprising a phospholipid membrane wherein the phospholipid membrane comprises a recombinant transmembrane protein and a fusogenic peptide provided herein. In some aspects, the disease is cancer. In certain aspects, the transmembrane protein is connexin and the vesicle enhances or restores cellular gap junction communication. In other aspects, the disease is cystic fibrosis. In some aspects, the vesicle enhances or restores endogenous CFTR function. In certain aspects, the vesicle is aerosolized prior to administration. In other aspects, the disease is a skin disease. In some aspects, the skin disease is Vohwinkel syndrome (VS), keratitis-ichthyosis-deafness (KID) syndrome, Bart-Pumphrey syndrome (BPS) or hystrix-like ichthyosis-deafness (HID) syndrome. In particular aspects, the vesicle is formulated for topical administration (e.g, a cream or lotion), transdermal administration, or subcutaneous injection.

In a further embodiment, there is provided a method of treating a disease or disorder in a subject comprising administering an effective amount of a therapeutic transmembrane protein, wherein the therapeutic transmembrane protein is provided in the phospholipid membrane of a vesicle.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIGS. 1A-1K: Giant plasma membrane vesicles (GPMVs) comprising connexin (referred to herein as Connectosomes) were harvested from donor cells. (A) Confocal fluorescence images. (B-D) Schematic of the Connectosome production process. GPMVs were extracted from donor cells overexpressing connexin 43-YFP (arrows) to produce Connectosomes, cell-derived lipid vesicle materials with embedded connexin 43-YFP connexons. (E) Multiple Connectosomes in a single field of view. (F-H) GPMVs (arrows) were extracted from donor cells treated with CRO dye to produce CRO dye-loaded Connectosomes. (I) Multiple CRO dye-loaded Connectosomes in a single field of view. (J) Histogram of Connectosome diameters. 154 Connectosomes were measured. (K) A calibration curve of YFP fluorescence was generated to determine the YFP content of the Connectosomes. All scale bars 20 μm except for (D) and (H) which are 2 μm. Images in (C) and (G) intentionally saturated to show Connectosome formation.

FIGS. 2A-2H: Connectosomes contained functional connexons. Confocal fluorescence images. (A) Connectosomes retained CRO dye in a solution of 2 mM Ca²⁺ (top), but released dye when Ca²⁺ was removed (bottom). (B) Percentage of Connectosomes releasing dye+/−Ca²⁺. The error bars represent the standard deviations of 3 independent trials; at least 54 Connectosomes analyzed per trial. (C) Schematic illustrating connexon-dependent molecular exchange. (D) GPMVs derived from MDA-MB-231 cells retained CRO dye in a solution of 2 mM Ca²⁺ (top), as well as when Ca²⁺ was removed (bottom). (E) Percentage of MDA-MB-231 GPMVs releasing dye+/−Ca²⁺. The error bars represent the standard deviations of 3 independent trials, at least 36 Connectosomes analyzed per trial. (F) Connectosomes excluded Atto 594 in 2 mM Ca²⁺ (top), but filled with dye when Ca²⁺ was removed (bottom). (G) Percentage of Connectosomes including dye+/−Ca²⁺. The error bars represent the standard deviations of 3 independent trials, at least 51 Connectosomes analyzed per trial. (H) The Atto 594 dye within Connectosomes (top) was photobleached (middle) in the absence of Ca²⁺. The Connectosomes refilled with dye within 75 seconds after the laser illumination was stopped (bottom). Scale bars: 2 μm. Asterisks represent statistically significant differences (two-tailed t-test, p<0.001).

FIGS. 3A-3E: Connectosomes delivered dye to the cellular cytoplasm. Brightfield and confocal fluorescence images. (A) Schematic. (B) Two Connectosomes (arrows) delivering CRO dye to the cellular cytoplasm. (C) Flow cytometry histograms showing CRO dye fluorescence for each recipient cell condition. The dotted line, drawn at the peak of the fluorescence histogram for cells receiving CRO dye-loaded Connectosomes, is used as a threshold in (E). Each curve represents 3 independent, concatenated trials, 10,000 cells analyzed per trial. (D) Average recipient cell fluorescence for each condition. The error bars represent the standard deviations of 3 independent trials, 10,000 cells analyzed per trial. (E) Percentage of cells with fluorescence values above the threshold drawn in (C). The error bars represent the standard deviations of 3 independent trials, 10,000 cells analyzed per trial. Legend in (C) applies to (D-E). Scale bar: 10 μm. Asterisks represent statistically significant differences (two-tailed t-test, p<0.04 (D) and p<0.01 (E)). Image of Connectosome in (B) intentionally saturated to show intracellular dye accumulation.

FIGS. 4A-4J: Connectosomes substantially reduced the cytotoxic dose of doxorubicin. (A-C) GPMVs were extracted from donor cells treated with doxorubicin to produce doxorubicin-loaded Connectosomes. (D) Schematic illustrating doxorubicin release from Connectosomes. (E) Average Connectosome fluorescence calculated from flow cytometry data. Connectosomes released significant amounts of doxorubicin within 5 minutes of calcium removal. The error bars represent the standard deviations of 3 independent trials, at least 800 Connectosomes analyzed per trial. (F) Schematic illustrating the 3 modes of drug delivery tested. (G) Percentage of nonviable HeLa cells after free doxorubicin treatment, conventional liposomal doxorubicin treatment, or doxorubicin-loaded Connectosome treatment. All points were measured using a 7-AAD viability assay, except for the free doxorubicin 105 nM point, which was measured using a trypan blue viability assay, owing to interference of doxorubicin in the 7-AAD measurement at this high doxorubicin concentration. The error bars represent the standard deviations of at least 3 independent trials, at least 4,000 cells (7-AAD assay) or 93 cells (trypan blue) analyzed per trial. (H) Flow cytometry histograms showing 7-AAD fluorescence for cells receiving doxorubicin-loaded Connectosomes at increasing equivalent free doxorubicin (dox) concentrations. The dotted line represents the threshold fluorescence value above which cells were considered nonviable. Each curve represents 3 independent, concatenated trials, at least 4,000 cells analyzed per trial. (I) Percentage of nonviable cells determined using both trypan blue and 7-AAD viability assays. The error bars represent the standard deviations of at least 3 independent trials, at least 4,000 cells (7-AAD assay) or 93 cells (trypan blue assay) analyzed per trial. (J) Percentage of nonviable MCF-7 cells after free doxorubicin treatment or doxorubicin-loaded Connectosome treatment. All points were measured using a trypan blue viability assay. The error bars represent the standard deviations of 3 independent trials, at least 166 cells analyzed per trial. Scale bars: 2 μm. Asterisks represent statistically significant differences (two-tailed t-test, p<0.02). Image in (B) intentionally saturated to show doxorubicin-loaded Connectosome formation.

FIGS. 5A-5B: Exogenously-loaded Connectosomes encapsulated a membrane impermeable dye. (A) Connectosomes excluded CRO dye in a solution of 2 mM Ca²⁺ (top), but filled with dye when Ca²⁺ was removed (bottom). (B) Percentage of Connectosomes including dye+/−Ca²⁺. The error bars represent the standard deviations of 3 independent trials; at least 39 Connectosomes analyzed per trial. Scale bars: 2 μm. Asterisk represents statistically significant differences (two-tailed t-test, p<0.002).

FIG. 6: Connectosomes (arrows) delivered dye to the cytoplasm. Brightfield and confocal fluorescence images. Scale bars: 20 μm. Connectosomes intentionally saturated to show intracellular dye accumulation.

FIGS. 7A-7E: Connectosomes delivered CRO dye to the cytoplasm. (A-D) Side-scatter versus forward-scatter plots showing all events detected in 3 independent, concatenated trials for untreated cells (A) and cells treated with carbenoxolone (B), carbenoxolone+CRO dye-loaded Connectosomes (D), and CRO dye-loaded Connectosomes (D). The gate shown was used for analysis in FIG. 3. (E) Average recipient cell fluorescence for the ungated samples for each condition. These results show approximately the same trend and relative magnitudes as the data in FIG. 3, demonstrating that the result of the experiment does not depend on the choice of the gate. For each trial, at least 17,000 events were detected. At least 10,000 of these events fell within the gate and were analyzed in FIG. 3. The error bars represent standard deviations of 3 independent trials. M stands for million.

FIG. 8A-8B: Dye delivery is dependent on gap junction assembly. (A) Flow cytometry histograms showing CRO dye fluorescence for each recipient cell condition: (i) untreated cells, (ii) cells treated with CRO-loaded blebs from A549 cells lacking connexin expression, (iii) Connectosomes derived from HeLa cells stably expressing Cx43. Each curve represents 3 independent, concatenated trials, at least 10,000 cells analyzed per trial. The population of highly fluorescent cells (centered around 10⁶), which is only present for cells exposed to Connectosomes, represents the fraction of cells that received a substantial dose of CRO dye. In contrast, cells treated with CRO-loaded GPMVs derived from A549 cells do not have significantly greater fluorescence than untreated cells. (B) Average recipient cell fluorescence for each condition, normalized to the average recipient cell fluorescence for cells treated with Connectosomes. The error bars represent the standard deviations of 3 independent trials, at least 10,000 cells analyzed per trial. Legend in (A) applies to (B). Asterisk represents statistically significant differences (two-tailed t-test, p<0.003).

FIGS. 9A-9B: Connectosomes contained doxorubicin. (A) A representative fluorescence spectrum of doxorubicin-loaded Connectosomes, compared to empty Connectosomes. Connectosomes were washed to remove free doxorubicin from solution. (B) A calibration curve of doxorubicin fluorescence in solution was generated by plotting the peak of the fluorescence spectrum for each concentration of doxorubicin dissolved in aqueous solution. A line was fit to this curve and the doxorubicin content of the Connectosomes was estimated by calculating the doxorubicin concentration corresponding to the measured fluorescence. The error bars represent the standard deviations of 3 independent trials.

FIGS. 10A-100: Thresholds for 7-AAD viability assay. (A-O) Flow cytometry histograms showing 7-AAD fluorescence histograms for cells with and without 7-AAD for each condition. Legend in (A) applies to (B-O). The dotted line represents the threshold fluorescence value above which cells were considered nonviable, for untreated cells (A), and for cells treated with 100 nM (B), 1 μM (C), and 10 μM (D) free doxorubicin, for cells treated with empty Connectosomes (E) and doxorubicin-loaded Connectosomes at equivalent doxorubicin concentrations of 15 nM (F), 150 nM (G), 400 nM (H), and 1.5 μM (I), and for cells treated with liposomal doxorubicin at equivalent doxorubicin concentrations of 10 nM (J), 100 nM (K), 1 M (L), 10 μM (M), 100 μM (N), and 1 mM (0). Each curve represents 3 independent, concatenated trials, at least 4,000 cells analyzed per trial.

FIGS. 11A-11B: Doxorubicin-loaded Connectosomes were cytotoxic to HeLa cells. Brightfield images. (A) Untreated, control HeLa cells. (B) Cells after treatment with doxorubicin-loaded Connectosomes at an equivalent doxorubicin dose of 400 nM. Scale bars: 200 μm.

FIGS. 12A-12J: Connectosomes were harvested from donor cells and contained functional connexons able to interact with recipient cells. (A) Schematic of Connectosomes production process. B) In sequential order: Confocal images of donor cells expressing CX43-YFP, cell during blebbing, collected intact blebs, and extruded Connectosomes. Scale bar correspond to 20 μm. (C) In sequential order: Confocal images of donor cells expressing CX43-YFP labeled with Texas Red DHPE, labeled cell during blebbing, collected intact blebs, and extruded labeled Connectosomes. Scale bar correspond to 20 μm. (D) Schematic illustrating Connectosomes calcium-dependent molecular gating: Connectosomes retained CRO dye in 2 mM Ca²⁺ (top), but released dye when Ca²⁺ was removed (bottom). Scale bar correspond to 2 μm. (E) GPMVs derived from A549 cells retained CRO dye in 2 mM Ca²⁺ (top), as well as when Ca²⁺ was removed (bottom). Scale bar correspond to 2 μm. (F) Percentage of releasing dye in the absence of Ca²⁺, (A) A549-derived blebs, (B) Connectosomes. Data collected in triplicates and standard deviation reported. Asterisks represent statistically significant differences (two-tailed t-test, P<0.01). (G) Schematic of Connectosomes (containing high concentration of CX43) and HeLa GPMVs (containing reduced amounts of CX43) interacting with different extent with recipient metastatic cancer cells. (H) Confocal images of recipient cells incubated for 4 hours with extruded Connectosomes. Overlaid bright field and fluorescent channel. Scale bar correspond to 10 μm. (I and J) Flow cytometry results. Data collected in triplicates and standard deviation reported. Asterisks represent statistically significant differences (two-tailed t-test, p<0.05).

FIGS. 13A-13D: Connectosomes decreased metastatic cancer cell migration potential in a transwell migration assay set up. (A) Schematic of the experimental set up. In sequential order: transwell membrane insert, MDA-MB-231 cells seeded on the upper compartment, MDA-MB-231 cells migrated through the membrane, filter collected and stained to assess the number of cell migrated. (B) Migration assay with cells treated with extruded GPMVs. Concentrations are expressed as a ratio between Connectosomes: one cell. Data were collected at least in triplicates and standard deviation is reported. Data were analyzed and compared with One-way Anova with post-hoc Tukey HSD (* for P=0.05). (C) Representative images of the membrane filters with the migrated cells, stained with crystal violet stained cells. Scale bar correspond to 250 μm. (D) MTT viability assay. MDA-MB cells were treated for 24 hours with either process or unprocessed Connectosomes at 2 different concentrations. Experiment was performed with n≧6, standard deviation was calculated with excel.

FIGS. 14A-14D: Connectosomes decreased metastatic cancer cell migration potential in a scratch assay set up. (A) Schematic of the experimental set up and representative images of the scratch at different time points. Scale bar correspond to 250 μm. (B) Results for extruded GPMVs expressed as: normalized scratch width=wₜ_{ₓ}/wₜ_{ₒ}, where Wₜ_{ₒ} is the width of the scratch at time zero and Wₜ_{ₓ} is the width of the scratch a subsequent time points. Asterisks represent statistically significant differences (two-tailed t-test, P<0.05). (C) Schematic of the hypothesized mechanism of action of the Connectosomes in metastatic recipient cancer cells. (D) Scratch assay performed on MDA-MB-231 cells treated with unprocessed GPMVs. Measurements were performed in triplicates, standard deviation was calculated with excel.

FIGS. 15A-15B: (A) Schematic. (B) Daily addition of Connectosomes increases the resistivity of A549 cell monolayers by reinforcing the defective connexin-based gap junction network among tumor cells.

FIGS. 16A-16C: Multi-functional targeting proteins expressed by the donor cells can be harvested through the extraction of GPMVs. (A) Cartoon schematics showing the architecture of the targeting protein, consisting of the intracellular and transmembrane domain of transferrin receptor (Tf-R), an eGFP, a long stretch of intrinsically disordered amino acids (289 aa) and an EGF ligand domain. (B) Confocal images of live CHO cells transiently expressing the EGF targeting protein and incubated with red ATTO 594-labeled antibodies against EGF. The green fluorescent cell is an example of a cell expressing the targeting protein. The dotted line shows a cell with little or no expression, which clearly does not recruit the antibody. (C) Cartoon schematics of extraction of GPMVs (left) and a confocal image of a CHO cell stably expressing the targeting protein undergoing GPMV extraction (right). The arrowheads point to the growing GPMVs from this donor cell plasma membrane surface. The image is intentionally saturated to show GPMV formation. (D) Donor cells after GPMV extraction (left and middle) have similar morphological appearance compared to healthy cells. Hoechst 33342 staining illustrated that the nuclei remained intact after the extraction process. All scale bars represent 10 μm.

FIGS. 17A-17E: GPMVs extracted from the plasma membranes of donor cells display functional targeting proteins on their surfaces at a high density. (A) Confocal images of GPMVs derived from CHO cells transiently expressing the EGF targeting protein and incubated with red-labeled antibodies against EGF. Fluorescent GPMV displays the targeting protein while the brightfield image shows three other GPMVs that do not display the targeting protein, presumably because they came from cells with low expression levels. The GPMVs that do not display the targeting protein clearly did not recruit the antibody. Scale bar represents 10 μm. (B) A calibration curve of GFP fluorescence. A linear fit to the curve was used to calculate the GFP content of a solution of GPMVs based on the intensity of eGFP fluorescence of the solution. The measured average of GPMV brightness represents 5 independent trials normalized to 2×10⁷ GPMVs and the error bars represent the standard deviation. 7D12 protein is an alternative targeting protein developed using a single chain variable domain only antibody, otherwise known as a nanobody, against EGFR as the ligand. (C) Confocal z-stack images of vesicles were taken and the frame where the vesicles settled on the glass coverslip and appeared as a solid circle of relatively uniform intensity was chosen for analysis. The average fluorescence intensity of the vesicle was determined from the intensity profile (shown in white). Scale bar represents 10 m. (D) Copies of targeting proteins per square micrometer. The histogram shows the brightness distribution of 43 GPMVs. (E) Transmission electron micrographs of plasma membrane vesicles (PMVs) showed that they have similar morphology to other liposomal particles.

FIGS. 18A-18D: PMVs displaying targeting proteins bound to EGFR-expressing cells. (A) Cartoon schematics of the targeting proteins. (B) Confocal images of HeLa cells transiently overexpressing mRFP-tagged EGFR extensively recruited EGF-PMVs. (C) Cells from the same culture dish, which had low levels of mRFP-tagged EGFR expression, recruited the PMVs to a much lesser extent. Both fluorescent images from B and C are under identical brightness and contrast setting. The brightfield image was overlaid to show the cell. (D) Dosage response curve for EGF-PMVs binding to MDA-MB-468 cells, a cell line with high endogenous EGFR expression. Each point is the average of 3 independent trials and the error bars show the standard deviation. At low PMV to cell ratios the peak shifts were small and variable owing to variations in the cellular autofluorescence. However as the PMV to cell ratio increased, a clear correlation with increasing peak shift was observed. Example histograms from flow cytometry analysis showing the shift in GFP-channel fluorescence upon exposure of the cells to PMVs. From top to bottom, the histograms correspond to 100 PMVs per cell, 600 PMVs per cell and 1300 PMVs per cell respectively. The p values are derived from a one-tailed unpaired t-test on the mean fluorescence values with (+ vesicle) and without vesicles (− vesicle). All scale bars, 10 μm.

FIGS. 19A-19C: PMV binding to cells is correlated with cellular expression of EGFR. (A) Breast cancer cell lines with increasing EGFR expression level recruit increasing densities of EGF-PMVs. The green fluorescent images are maximum intensity Z projects. (B) Example histograms from flow cytometry analysis showing an increase in the GFP-channel fluorescence upon exposure of cells to EGF and 7D12-PMVs. Breast cancer cells were incubated with PMVs at a concentration of 1300 PMVs per cell. The p values are derived from a one-tailed unpaired t-test on the mean fluorescence values with (+ vesicle) and without vesicles (− vesicle). (C) Mean fluorescence analysis quantified using flow cytometry, showing PMV binding vs. EGFR expression level. The relative EGFR expression level of these cancer cells was also quantified using flow cytometry. The error bars represent the standard deviation of 3 independent measurements. All scale bars, 10 μm.

FIGS. 20A-20D: Targeted PMVs provide a general strategy for specific binding to any cell-surface protein, including GFP-labeled receptors. (A) Cartoon schematics showing the GFPnb targeting protein binding to soluble eGFP. Here the ligand, GFPnb, is a nanobody against GFP. (B) Confocal images of CHO wild type cells transiently expressing the GFPnb targeting protein. The single bright cell in the mRFP fluorescent channel is the only cell in this field of view that expresses the GFPnb targeting protein (compare brightfield and mRFP fluorescent images). As expected, only this cell recruits soluble eGFP from solution, demonstrating specific binding. (C) Confocal images of a GPMV derived from CHO cells stably expressing the GFPnb targeting protein. Incubation of GPMVs with soluble eGFP shows binding. (D) Display of the GFPnb targeting protein is correlated with recruitment of soluble eGFP based on fluorescence intensity analysis. A total of 39 GPMVs were analyzed, using two measurements per GPMV on opposite edges. All scale bars, 10 μm.

FIGS. 21A-21E: GFPnb-PMVs bind to eGFP expressing cells with high specificity. (A) Cartoon schematics showing competitive binding assay. Only cells that express GFP-tagged receptors on their surfaces are expected to recruit GFPnb-PMVs. (B) The specificity GFPnb-PMV binding to cells was evaluated by co-culturing GFP negative and GFP positive cells in a 1:1 ratio and then exposing them simultaneously (in the same culture dish) to GFPnb-PMVs. GFP positive cells recruited GFPnb-PMVs in substantially greater quantities. The red fluorescent image is a maximum intensity Z projection. (C) Flow cytometry analysis of GFPnb-PMV binding to the co-cultured cells. Top: The fluorescence signals of GFP positive and negative cells were used to set separate gates to distinguish these two co-cultured populations of cells. Bottom: The overlay of recipient cell fluorescence with and without GFPnb-PMVs. Only the GFP positive cells (right) have a clearly detectable fluorescence shift upon PMV binding. (D) Mean fluorescence increase owing to GFPnb-PMV binding. (E) GFPnb PMVs were used to demonstrate the preservation of transmembrane protein topology. GPMVs extracted from CHO cells transiently express transmembrane receptors with an extracellular GFP recruited GFPnb-PMVs (top) while those from CHO cells transiently express EGFR with an intracellular GFP did not (bottom). The line scans show the intensity of mRFP signal from GFPnb-PMVs. These images were taken under same camera setting and displayed using identical brightness and contrast for direct comparison. All scale bars, 10 μm.

FIGS. 22A-22B: (A) GPMVs containing GFP-labelled CFTR transmembrane protein were developed (A) and successfully nebulized. Average diameter of CFTR PMVs after nebulization is 406.6 nm. (B) A binding assay was performed to show the retention of biological activity of nebulized EGF-PMVs to cells expressing EGFR. Untreated cells and cells treated with extruded GPMVs were used as control.

### DESCRIPTION OF ILLUSTRATIVE EMBODIMENTS

The present disclosure overcomes challenges associated with current technologies by providing methods and compositions for the treatment of diseases with mutated or dysfunctional transmembrane proteins by the delivery of recombinant transmembrane proteins. The inventors have discovered that one method of delivering recombinant transmembrane proteins is by vesicles with recombinant transmembrane proteins embedded in the phospholipid membrane. The vesicles can additionally comprise a fusogenic peptide that allows fusion of the vesicle membrane with the membrane of a recipient cell so that the transmembrane protein is delivered to the recipient cell membrane.

In one method, the vesicles are harvested from the plasma membranes of donor cells genetically modified to express the recombinant transmembrane protein. In particular, donor cells can be genetically manipulated to overexpress the transmembrane protein and induced to expel portions of their plasma membranes through cellular blebbing to form vesicles. In other aspects, the genetically manipulated donor cells can be induced to precipitate vesicles (e.g., exosomes) by the addition of a polymer.

Specifically, the transmembrane proteins can be connexins which form functional gap junction channels. Accordingly, these vesicles with gap junctions can deliver the gap junction proteins to recipient cells, such as cancer cells, to enhance gap junction communication. The vesicles with gap junctions can also form controllable gap junction interfaces with cells enabling direct delivery of molecular cargoes to the cytoplasm. This method of delivery can be used to efficiently deliver therapeutics such as chemotherapeutics directly to cancer cells.

Additionally, vesicles with the transmembrane protein Cystic Fibrosis Transmembrane Conductance Regulator (CFTR) embedded in the phospholipid membrane are provided. Thus, in certain aspects, methods of treatment are provided for treating Cystic Fibrosis in a subject by administering the vesicles with CFTR embedded in the membrane provided herein to deliver functional CFTR protein to lung cells with mutated CFTR protein. In some aspects, the vesicles are aerosolized before administration for direct delivery to cells in the lungs.

Thus, methods of treating a disease with a mutated or dysfunctional transmembrane protein by the administration of vesicles with recombinant transmembrane proteins and a fusogenic peptide embedded in the membrane are provided herein. There are numerous diseases with mutated or dysfunctional transmembrane proteins that can be treated by the methods disclosed herein such as cancer, cystic fibrosis, CNS diseases, metabolic disorders, asthma, atherosclerosis, orphan diseases, and skin diseases.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

“Liposomes” are defined herein as closed vesicles composed of a lipid assembly in a membrane form and an aqueous phase within the membrane. The liposome that can be used generally has a size of 20 nm to 100 jam, preferably 200 nm to 10 am. Liposomes can have a single lipid bilayer (i.e., unilamellar liposomes) or multiple lipid bilayers (i.e., multilamellar liposomes). Each bilayer surrounds, or encapsulates, an aqueous compartment. Given this encapsulation of aqueous volume within a protective barrier of lipid molecules, liposomes are able to sequester encapsulated molecules, such as nucleic acids, away from the degrading effects of factors, such as nuclease enzymes, present in the external environment.

The term “phospholipid” is used herein to refer a class of lipids that generally consist of two hydrophobic fatty acid tails and a hydrophilic head consisting of a phosphate group, wherein the two components are joined together by a glycerol molecule. Phospholipids can form phospholipid bilayers because of their amphiphillic characteristic.

“Vesicles” are referred to herein as non-living fluid-filled sacs enclosed by a phospholipid bilayer. Vesicles preferably have a diameter of about 30 nm to about 100 μm. Larger vesicles may have a diameter is about 1 μm to 100 μm, and smaller vesicles (e.g., exosomes) may have a diameter of 30 nm to 150 nm.

“Giant plasma membrane vesicles (GPMVs)” are referred to herein as liposomes produced by cellular blebbing, that possess a phospholipid bilayer, are generally spherical and can have a diameter of about 1 μm to about 100 μm. The GPMVs can be processed to have smaller diameters such as by extrusion to produce “plasma membrane vesicles (PMVs)”, such as around 30-100 nm. The GPMVs or PMVs are also known as “blebs” which are bud-like protrusions formed in the cell wall, outer membrane, cytoplasmic and/or plasma membrane of a cell. When cultured under selected conditions described herein the GPMVs break away from the whole cell into the medium.

The terms “Connectosomes” or “Gap junction vesicles (GJVs)” refer to GPMVs or PMVs which comprise high copy numbers of connexin proteins. The terms “Vesicles”, “Connectosomes”, “Gap junction vesicles (GJVs)”, “Giant plasma membrane vesicles (GPMVs)”, “plasma membrane vesicles (PMVs)” and “blebs” are used throughout the present disclosure.

“Exosomes” are small secreted vesicles (e.g, about 30-150 nm) which may contain, or have present in their membrane, nucleic acids, proteins, or other biomolecules.

A “blebbing buffer” is defined herein as a buffer which induces the production of plasma membrane vesicles from donor cells through blebbing also known as vesiculation.

The term “cancer cell” denotes a cell that demonstrates inappropriate, unregulated proliferation. A “human” tumor is comprised of cells that have human chromosomes. Such tumors include those in a human patient, and tumors resulting from the introduction into a non-human host animal of a malignant cell line having human chromosomes into a non-human host animal.

The term “recombinant” or “engineered” when used with reference to a cell indicates that the cell replicates or expresses a nucleic acid or expresses a peptide or protein encoded by a nucleic acid, whose origin is exogenous to the cell. Recombinant cells can express nucleic acids that are not found within the native (non-recombinant) form of the cell.

Recombinant cells can also express nucleic acids natively expressed in the cell, wherein the nucleic acids are reintroduced into the cell by artificial means in order to alter the expression of that gene.

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, the term refers to a cell that was isolated and subsequently introduced to other cells or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from where it would be in natural cells, or is otherwise flanked by a different nucleic acid sequence than that found in nature.

By “expression construct” or “expression cassette” is meant a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

A “vector” or “construct” (sometimes referred to as a gene delivery system or gene transfer “vehicle”) refers to a macromolecule or complex of molecules comprising a polynucleotide to be delivered to a host cell, either in vitro or in vivo.

A “plasmid,” a common type of a vector, is an extra-chromosomal DNA molecule separate from the chromosomal DNA that is capable of replicating independently of the chromosomal DNA. In certain cases, it is circular and double-stranded.

A “gene,” “polynucleotide,” “coding region,” “sequence,” “segment,” “fragment,” or “transgene” that “encodes” a particular protein, is a nucleic acid molecule that is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

A “fusogenic” peptide as defined herein is a peptide that is capable of interacting or fusing with a recipient cell membrane in a way that permits delivery of the transmembrane protein to the recipient cell membrane.

As used herein the term “targeting ligand” or “targeting molecule” refers to any suitable targeting moiety that can be either chemically conjugated to, or directly associated/complexed with, the vesicles provided herein. In embodiments where the ligand is directly associated/complexed with, but not chemically conjugated to the cationic liposomes, no linker, spacer or other bridging molecule is used to complex the ligands to the liposomes.

A “membrane protein” is a protein positioned in a membrane. Certain membrane proteins, particular those proteins that are naturally found in membranes, will typically comprise, or are modified to comprise, a membrane anchoring domain or transmembrane domain, which serve to “anchor” the protein in the membrane dues to the presence of hydrophobic amino acids.

A “transmembrane protein” is a type of membrane protein spanning from one side of a membrane through to the other side of the biological membrane in which it is embedded.

A “recombinant transmembrane protein” is a transmembrane protein engineered to be embedded in a phospholipid membrane. Generally, the protein is encoded by a nucleic acid, whose origin is exogenous to a cell, which is introduced into the cell by artificial means.

The term “connexin” denotes a family of genes and gene products wherein the gene products are structural subunits of gap junctions, and variants thereof. “Connexin” further denotes nucleic acid sequences and their gene products, wherein the gene products are recognized by antibodies that specifically bind to a connexin protein and, when expressed in cells, may be present in gap junctions.

An “expression vector” is an artificial nucleic acid molecule into which an exogenous nucleic acid molecule encoding a protein can be inserted in such a manner so as to be operably linked to appropriate expression sequences that direct the expression of the exogenous nucleic acid molecule.

The term “autologous” is used herein to refer to cells that are isolated from the patient in need thereof.

A “chemotherapeutic” drug as used herein refers to those drugs commonly used in the treatment of cancer. These agents act through an apoptotic mechanism of cell death. Each of the drugs can differ in the mechanism by which the cells enter apoptosis.

The phrase “effective amount” means a dosage of a drug or agent sufficient to produce a desired result. The desired result can be subjective or objective improvement in the recipient of the dosage, a decrease in tumor size, a decrease in the rate of growth of cancer cells, a decrease in metastasis, or any combination of the above.

“Pharmaceutically acceptable carriers” as used herein are those media generally acceptable for use in connection with the administration of lipids and liposomes, including liposomal bioactive agent formulations, to animals, including humans.

As used herein, the term “aerosols” refers to dispersions in air of solid or liquid particles, of fine enough particle size and consequent low settling velocities to have relative airborne stability (See Knight, V., Viral and Mycoplasmal Infections of the Respiratory Tract. 1973, Lea and Febiger, Phila. Pa., pp. 2). “Liposome aerosols” consist of aqueous droplets within which are dispersed one or more particles of liposomes or liposomes containing one or more medications intended for delivery to the respiratory tract of man or animals (Knight, V. and Waldrep, J. C. Liposome Aerosol for Delivery of Asthma Medications; see also In Kay, B., Allergy and Allergic Diseases, 1997, Blackwell Publications, Oxford, England, Vol. I pp. 730-741). The size of the aerosol droplets defined for this application are those described in U.S. Pat. No. 5,049,338, namely mass median aerodynamic diameter (MMAD) of 1-3 μm with a geometric standard deviation of about 1.8-2.2. Based on the studies disclosed by the present disclosure, the liposomes may constitute substantially all of the volume of the droplet when it has equilibrated to ambient relative humidity.

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

### II. TRANSMEMBRANE PROTEINS

Functional, therapeutic transmembrane proteins are delivered to cells by the methods disclosed herein. Membrane proteins consist, in general, of two types, peripheral membrane proteins and integral membrane proteins. Integral membrane proteins can span the two layers of a lipid bilayer membrane. Thus, such proteins may have extracellular, transmembrane, and intracellular domains. Extracellular domains are exposed to the external environment of the cell, whereas intracellular domains face the cytosol of the cell. The portion of an integral membrane protein that traverses the membrane is the transmembrane domain. Transmembrane domains traverse the cell membrane often by one or more regions comprising typically 15 to 25 hydrophobic amino acids which are predicted to adopt an alpha-helical conformation.

Other membrane proteins that are within the scope of the present disclosure and include but are not limited to channels (e.g., potassium channels, sodium channels, calcium channels), pores (e.g., nuclear pore proteins, water channels), ion and other pumps (e.g., calcium pumps, proton pumps), exchangers (e.g., sodium/potassium exchangers, sodium/hydrogen exchangers, potassium/hydrogen exchangers), electron transport proteins (e.g., cytochrome oxidase), enzymes and kinases (e.g., protein kinases, ATPases, GTPases, phosphatases, proteases), structural/linker proteins (e.g., Caveolins, clathrin), and adapter proteins (e.g., TRAD, TRAP, FAN).

A. Connexins

Direct intercellular communication mediated by gap junctions (GJs) is a hallmark of normal cell and tissue physiology. It is established by GJ channels that bridge apposing plasma membranes of neighboring cells. In addition, GJs significantly contribute to physical cell-cell adhesion and these cellular functions require precise modulation. GJ channels are double membrane proteins structures that mediate direct cell-cell communication by allowing the passage of molecules up to about 1 kDa from one cell to the other. Typically, GJs represent arrays of hundreds of thousands of densely packed channels, each one assembled from two half-channels (i.e., connexons) that dock head-on in the extracellular space to form the channel arrays that link neighboring cells together. Connexons are composed of six polytopic transmembrane protein subunits, termed connexins. Connexins comprise a large gene family predicted to consist of 20 isoforms in humans alone. The ability to modulate (up- and downregulate) the level of GJ-mediated intracellular communication, and of physical cell-cell adhesion is as vitally important as the basic ability of GJ formation itself; and is for example crucial for many physiological and pathological conditions, including cell migration during development and wound healing, mitosis, apoptosis, leukocyte extravasation, ischemia, hemorrhage, edema, and cancer metastasis.

GJ channels are assembled from a ubiquitously expressed class of four-pass transmembrane proteins, termed connexins, with connexin 43 (Cx43) being the most abundantly expressed connexin type. Six connexin polypeptides oligomerize into a ring to form a hexameric structure with a central hydrophilic pore, called hemi-channel or connexon. Once trafficked to the plasma membrane, two connexons, one provided by each of two neighboring cells, dock head-on in the extracellular space to form the complete, tightly sealed to the outside, transmembrane GJ channel. Recruitment of additional GJ channels along the outer edge then enlarges the channel plaques, while simultaneous removal of older channels from plaque centers balances GJ channel turnover (Lauf et al., 2002).

A wide range of connexins known in the art can be used as the connexin incorporated in the vesicles of the present disclosure. For example, the connexin can be connexin 46, connexin 43, connexin 37, connexin 40, connexin 50, connexin 32, connexin 26, connexin 31, connexin 31.1, connexin 45, connexin 30, connexin 36, connexin 62, connexin 31.9, or connexin 40.1.

In certain aspects, the connexin is Connexin 43. Connexin 43 (Cx43) is a member of the gap junction (GJ) protein family, connexins, which consist of at least 15 homologous proteins ranging in size from 26 to 56 kilodaltons (kDa). Cx43 is widely expressed, and like other gap junction proteins, forms intercellular plasma membrane channels that allow ions and small molecules, such as but not limited to molecules of less than 1 kDa, to pass through. Cx43 plays an important role in tissue homeostasis, embryonic development, cell proliferation and differentiation. Brain and heart tissues are found to particularly express Cx43 (Yamasaki et al., 1996). Cx43 knockout mice die at birth due to cardiac malformations, suggesting a critical role of cx43 in development and in the fundamental physiology of multicellular organisms (Reaume et al., 1995).

B. Cystic Fibrosis Transmembrane Conductance Regulator (CFTR)

Cystic Fibrosis (CF) is caused by a homozygous mutation in the cystic fibrosis transmembrane conductance regulator (CFTR) gene. This gene codes for a chloride ion channel important in multiple functions including mucus homeostasis. Mutations of this gene, such as ΔF508, result in defects in this membrane channel protein, such as mis-folding (Riordan, 2008). These defects lead to the production of thick mucus secretions, which results in several complications including chronic bacterial lung infections and associated lung inflammation. CF related lung disease is the primary cause of the shortened life expectancy of this genetic disorder.

Inhaled drugs, including antibiotics and mucus thinners, are the standard of care for CF because they quickly and easily reach the airway; however, no cure is currently available. Due to the single genetic cause of CF, gene therapy has been extensively explored as a potential treatment (Griesenbach et al., 2012). Both viral and non-viral gene therapy approaches have been tried. Several intrinsic drawbacks of virus mediated-gene therapies, including limited opportunity for repeated administrations due to acute inflammatory response and delayed cellular immune responses, have precluded its translation into clinical use. Non-viral methods, although less immunogenic, are generally less efficacious than the viral methods, and in many cases, the gene expression is short-lived (Al-Dosari et al., 2009). Thus, there is a need for novel approaches for CF therapy that may replace the abnormal CFTR protein.

### III. TRANSMEMBRANE PROTEIN DELIVERY

Vesicles with recombinant transmembrane proteins are provided by the methods disclosed herein for the treatment of conditions or diseases in subjects in need thereof. The condition or disease is a disease in which there is a mutated or dysfunctional transmembrane protein. Thus, the vesicles of the present disclosure can deliver therapeutic transmembrane proteins to the plasma membrane of cells with the corresponding mutated or dysfunctional transmembrane protein in the subject.

Other methods of delivering recombinant transmembrane proteins for the treatment of conditions or disease in subjects can also be used. In one method, the recombinant transmembrane protein may be administered as a liposome and lipid complex composition such as described in U.S. Pat. No. 5,549,910 and U.S. Pat. No. 5,616,334, both incorporated herein by reference. In another method, the recombinant transmembrane proteins are delivered as nanodiscs such as described in U.S. Patent Publication No. US20130165636 and Denisov et al., Biochem. Biophys. Acta., 1814: 223-229, 2010, both incorporated herein by reference.

A. Vesicle Formation

1. Membrane Blebbing

Membrane blebbing is involved in multiple physiological processes including cell motility, mitosis, chemotaxis, viral entry, and maintenance of cell polarity, morphology and mechanical homeostasis. Plasma membrane blebs form when attachments between the plasma membrane and the cytoskeleton are disrupted (Mahadevan, 2008). Embodiments of the present disclosure provide methods for the production of vesicles with embedded transmembrane proteins through cellular blebbing to maintain the directional insertion and function of transmembrane proteins.

In one embodiment, the giant plasma membrane vesicles (GPMVs) are formed by contacting a donor cell with a blebbing buffer to induce vesicle production. A variety of donor cells may be used to prepare the GPMVs. The cells or cell lines may grow attached to a surface or free in growth media. Cells can be from any organism, preferably from mammals, such as humans. For example, the donor cells are human epithelial cells, endothelial cells or suspension cell lines that grow in high density cultures (e.g., non-adherent CHO cells). Alternatively, the donor cells are autologous cells. In one embodiment, the cells used to make plasma membrane vesicles are cells associated with a disease state, e.g., cancer. In another embodiment, the cells are transformed or transfected to yield a protein of interest. In exemplary embodiments, the protein of interest is one or several transmembrane proteins.

The blebbing buffer is used to induce donor cells to produce vesicles. The blebbing buffer can comprise a sulfhydryl blocking agent, buffer compound, a reducing agent and at least one salt. Preferably, the sulfhydryl blocking agent is paraformaldehyde (PFA). Common buffer compounds known in the art can be used including TAPS, Bicine, Tris, Tricine, TAPSO, HEPES, TES, MOPS, PIPES, Cacodylate, SSC, MES, and Succinic acid. For example, the buffer is an amine-type buffer such as Tris. Common reducing agents known in the art can be used including dithiothreitol, lithium aluminum hydride, and diborane. In an exemplary formulation, the blebbing buffer comprises calcium chloride (CaCl₂), HEPES, sodium chloride (NaCl), PFA and dithiothreitol (DTT). Alternatively, the blebbing buffer can comprise formaldehyde, DMSO, latrunculin or N-ethylmaleimide. Usually, the donor cells are washed to remove culture medium prior to contacting the cells with the blebbing buffer. It is contemplated that other methods of producing blebs known in art can also be used (e.g., Yanase et al., Biochem J., 425: 179-193, 2010; Ruan et al., PLoS One, 10, 2015; Tinevez et al., PNAS, 106: 18581-18586, 2009; Chharas et al., Nature, 435, 2005; Baumgart et al., PNAS, 104: 3165-3170, 2007; all incorporated herein by reference).

A salt is a compound formed by the interaction of an acid and a base. Salts known in the art can be used with the present disclosure. Particular salts can be acetate (e.g., sodium acetate), citrate (e.g., sodium chloride), sulphate (e.g., sodium sulphate), or a potassium salt.

The vesicles can be purified by various methods known in the art such as filtering, density gradient centrifugation or dialysis. For clinical use, the vesicles can be purified by multi-step centrifugations at increasing speeds using density gradients combined with washing steps.

Various methodologies such as sonication, homogenization, French Press application and milling can be used to prepare vesicles of a smaller size from larger vesicles. Generally, extrusion (U.S. Pat. No. 5,008,050, incorporated herein by reference) can be used to size reduce vesicles, that is to produce vesicles having a predetermined mean size by forcing the vesicles, under pressure, through filter pores of a defined, selected size. Tangential flow filtration (WO89/008846, incorporate herein by reference) can also be used to regularize the size of vesicles, that is, to produce a population of vesicles having less size heterogeneity, and a more homogeneous, defined size distribution.

The vesicles produced by the methods disclosed herein can be populations of monodisperse vesicles. The vesicles can have a diameter from 5 μm to 25 nm, from 50 μm to 500 μm, from 100 μm to 200 μm or preferably from 5 μm to 25 μm. In some embodiments, the diameters of the vesicles are within about 20%, 15%, 10%, 5%, 4%, 3%, or 2% of each other.

2. Exosome Isolation

In some embodiments, the vesicles of the present disclosure are exosomes. A variety of methods known in the art for the isolation of exosomes (see, for example, Lane et al., Scientific Reports, 5, 2015; incorporated herein by reference in its entirety) can be used in the present disclosure.

A variety of donor cells may be used to prepare the exosomes. The cells or cell lines may grow attached to a surface or free in growth media. Cells can be from any organism, preferably from mammals, such as humans. For example, the donor cells are human epithelial cells, endothelial cells or suspension cell lines that grow in high density cultures (e.g., non-adherent CHO cells). Alternatively, the donor cells are autologous cells. In one embodiment, the cells used to make plasma membrane vesicles are cells associated with a disease state, e.g., cancer. In another embodiment, the cells are transformed or transfected to yield a protein of interest. In exemplary embodiments, the protein of interest is one or several transmembrane proteins.

In one method, donor cells can be contacted with a polymer to induce precipitation of exosomes (U.S. Patent Application Nos. 2013/0273544 and 2015/0104801; both incorporated herein by reference in their entirety). The polymer may be polyethylene glycol, dextran, dextran sulfate, dextran acetate, polyvinyl alcohol, polyvinyl acetate, or polyvinyl sulfate. After completion of the incubation of the sample with the polymer the precipitated exosomes may be isolated by centrifugation, ultracentrifugation, filtration or ultrafiltration. Exosomes may be further fractionated using conventional methods such as ultracentrifugation with or without the use of a density gradient to obtain higher purity. Sub-populations of exosomes may also be isolated by using other properties of the exosome such as the presence of surface markers. Surface markers which may be used for fraction of exosomes include but are not limited to tumor markers and MHC class II markers. MHC class II markers which have been associated with exosomes include HLA DP, DQ and DR haplotypes. Other surface markers associated with exosomes include CD9, CD81, CD63 and CD82

3. Recombinant Transmembrane Proteins

Nucleotide sequences encoding exogenous proteins, such as transmembrane proteins, can be introduced into the donor cells to produce membrane vesicles using common molecular biology techniques known to those of skill in the art. The necessary elements for the transcription and translation of the inserted nucleotide sequences may be selected depending on the cell chosen, and may be readily accomplished by one of ordinary skill in the art. A reporter gene which facilitates the selection of cells transformed or transfected with a nucleotide acid sequence may also be incorporated in the microorganism. (see, e.g., Sambrook et al. Molecular Cloning A Laboratory Manual, 2nd edition, Cold Spring Harbor Laboratory Press, 1989, for transfection/transformation methods and selection of transcription and translation elements, and reporter genes). Sequences which encode exogenous proteins may generally be obtained from a variety of sources, including for example, depositories which contain plasmids encoding sequences including the American Type Culture Collection (ATCC, Rockville Md.), and the British Biotechnology Limited (Cowley, Oxford England). For example, an expression vector for Connexin 43 such as Cx43-GFP (Fong et al., 2012) can be transfected into donor cells for the production of membrane vesicles over-expressing Connexin 43. In other aspects, an expression vector for CFTR such as pEGFP-C1 vector can be transfected into donor cells for the production of membrane vesicles that over-express CFTR.

One of skill in the art would be well-equipped to construct a vector through standard recombinant techniques (see, for example, Sambrook et al., 2001 and Ausubel et al., 1996, both incorporated herein by reference). Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors.

In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor-mediated endocytosis, and to integrate into host cell genomes and express viral genes stably and efficiently have made them attractive candidates for the transfer of foreign nucleic acids into cells (e.g., mammalian cells).

The use of plasmid- or liposome-based extra-chromosomal (i.e., episomal) vectors may be also provided in certain aspects of the present disclosure. Such episomal vectors may include, e.g., oriP-based vectors, and/or vectors encoding a derivative of EBNA-1. These vectors may permit large fragments of DNA to be introduced unto a cell and maintained extra-chromosomally, replicated once per cell cycle, partitioned to daughter cells efficiently, and elicit substantially no immune response.

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide.

Such components also may include markers, such as detectable and/or selection markers that can be used to detect or select for cells that have taken up and are expressing the nucleic acid delivered by the vector. Such components can be provided as a natural feature of the vector (such as the use of certain viral vectors that have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

Eukaryotic expression cassettes included in vectors useful in the present disclosure preferably contain (in a 5′-to-3′ direction) a eukaryotic transcriptional promoter operably linked to a protein-coding sequence, splice signals including intervening sequences, and a transcriptional termination/polyadenylation sequence.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Introduction of a nucleic acid, such as DNA or RNA, into cells to be programmed with the present disclosure may use any suitable methods for nucleic acid delivery for transformation of a cell, as described herein or as would be known to one of ordinary skill in the art. Such methods include, but are not limited to, direct delivery of DNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

4. Synthetic Methods

In other embodiments, the vesicles provided herein can be produced by synthetic methods known in the art. Liposomes can be produced by a variety of methods (for a review, see, e.g., Cullis et al. (1987)). Bangham's procedure (J. Mol. Biol. (1965)) produces ordinary multilamellar vesicles (MLVs). Lenk et al. (U.S. Pat. Nos. 4,522,803, 5,030,453 and 5,169,637), Fountain et al. (U.S. Pat. No. 4,588,578) and Cullis et al. (U.S. Pat. No. 4,975,282) disclose methods for producing multilamellar liposomes having substantially equal interlamellar solute distribution in each of their aqueous compartments. Paphadjopoulos et al., U.S. Pat. No. 4,235,871, discloses preparation of oligolamellar liposomes by reverse phase evaporation.

Unilamellar vesicles can be produced from MLVs by a number of techniques, for example, the extrusion of Cullis et al. (U.S. Pat. No. 5,008,050) and Loughrey et al. (U.S. Pat. No. 5,059,421). Sonication and homogenization cab be so used to produce smaller unilamellar liposomes from larger liposomes (see, for example, Paphadjopoulos et al. (1968); Deamer and Uster (1983); and Chapman et al. (1968)).

Liposomes may be produced by hydration and mechanical dispersion of dried lipoidal material in an aqueous solution. The lipoidal material can be phospholipids or other lipids, cholesterol and its derivatives or a variety of amphiphiles including macromolecules or mixtures of these. However, liposomes prepared this way are mixtures of all the types noted above, with a variety of dimensions, compositions and behaviours. This unpredictable variety leads to inconsistent measures of liposome properties and unreliable characterizations. To reduce the heterogeneity of mechanically dispersed liposomes, such dispersions may be filtered through a membrane filter (see FR 2298318A) exposed to sonication which decreases average liposome size. Under extensive sonication, occasionally populations of liposomes are reduced to small unilamellar vesicles, but the sonic process does not give homogeneous dispersions of larger vesicles and can degrade the complex lipids and other components of the liposomes. The single filtration step disclosed in FR 2298318A still provides relatively random size particles. The preparation of liposomes and their use in drug therapy has been previously described. See, for instance, U.S. Pat. No. 4,053,585; Geman Patent 2,532,317; Netherlands application 73/04133; and Biochemistry 16 (12) 2806 (1977).

Generally, those agents which are to compose the lipid membrane of the liposome, such as phospholipids, cholesterol and/or other biologically active or inactive amphiphiles, or macromolecules are mixed in an organic solvent such as ethers, chloroform, alcohol, etc. and then dried onto the interior surface of a vessel under a vacuum. As an example, phosphatidic acid L-alpha-lecithin and cholesterol are mixed into a solution of 7:3:1 chloroform: isopropanol: methanol respectively and vacuum dried. An aqueous solution of the drug is added to the dried lipids at a temperature above the phase transition temperature of the lipid mixture. In this example, a bis-anthracycline at 1 mg/ml in isotonic phosphate buffer is added and the solution rolled with the lipids for one hour to allow slow hydration.

The liposomes are then subjected to an extrusion under a pressure at a pressure such as of at least about 1170 bar through a small orifice. For example, the liposomes are extruded using a French Press and Pressure Cell (Aminco type) maintained at about 1170 bar during the entire extrusion. The extrusion at this pressure may be repeated for enhanced uniformity of liposome. The extrusion pressure, orifice size, and temperature can be used to control the size of the resulting vesicles and very uniform liposomes can be easily and reproducibly made by this process. Extrusion may be at pressures up to 2070 bar. Subsequent to the extrusion, the free untrapped drug can be removed readily by dialysis leaving a uniform, stable liposome population (European Patent No. EP0036676B2).

Liposomes can be composed of a variety of lipids, both amphipathic and nonamphipathic, obtained from a variety of sources, both natural and synthetic. Suitable liposomal lipids include, without limitation, phospholipids such as phosphatidylcholines (“PC's”), phosphatidylethanolamines (“PE's”), phosphatidylserines (“PS's”), phosphatidylglycerols (“PG's”), phosphatidylinositols (“PI's”) and phosphatidic acids (“PA's”). Such phospholipids generally have two acyl chains, these being either both saturated, both unsaturated or one saturated and one unsaturated; said chains include, without limitation: myristate, palmitate, stearate, oleate, linoleate, linolenate, arachidate, arachidonate, behenate and lignocerate chains.

Phospholipids can also be derivatized to the vesicles, by the attachment thereto of a suitable reactive group. Such a group is generally an amino group, and hence, derivatized phospholipids are typically phosphatidylethanolamines. The different moieties suited to attachment to PE's include, without limitation: acyl chains (WO98/16199), useful for enhancing the fusability of liposomes to biological membranes; peptides (WO98/16240), useful for destabilizing liposomes in the vicinity of target cells; biotin and maleimido moieties (U.S. Pat. Nos. 5,059,421 and 5,399,331, respectively), useful for linking targeting moieties such as antibodies to liposomes; and, various molecules such as gangliosides, polyalkylethers, polyethylene glycols and organic dicarboxylic acids (see, e.g., U.S. Pat. Nos. 5,013,556, 4,920,016 and 4,837,028).

The vesicles may be dehydrated, stored and then reconstituted such that a substantial portion of their internal contents are retained. Liposomal dehydration generally requires use of a hydrophilic drying protectant such as a disaccharide sugar at both the inside and outside surfaces of the liposomes' bilayers (see U.S. Pat. No. 4,880,635, incorporated herein by reference). This hydrophilic compound is generally believed to prevent the rearrangement of the lipids in liposomes, so that their size and contents are maintained during the drying procedure, and through subsequent rehydration. Appropriate qualities for such drying protectants are that they be strong hydrogen bond acceptors, and possess stereochemical features that preserve the intermolecular spacing of the liposome bilayer components. Alternatively, the drying protectant can be omitted if the liposome preparation is not frozen prior to dehydration, and sufficient water remains in the preparation subsequent to dehydration.

B. Vesicle Loading

The vesicles can be loaded with compounds such as drugs, metabolites, RNAi, peptides, or small molecules. Examples of the compounds can include, but are not limited to, compounds having a known function as an active drug ingredient, organic compounds, nucleic acids, peptides, and compounds having an unknown function. The donor cells can be treated with the compound before vesicle formation to incorporate the compound into the vesicle. Alternatively, the compound can be loaded into the vesicle via the gap junction incorporated in the vesicle membrane. Therefore, the intended substance has a molecular weight level that can pass through the connexon and is preferably, for example, of 2000 or lower in molecular weight. Chemical gating of gap junction channels such as Ca²⁺ concentration or pH can be used to load or release compounds from vesicles (Perachhia, 2004). In exemplary methods, the gap junctions can be opened in the presence of low calcium concentrations by the addition of EDTA and/or EGTA to remove calcium, and the gap junctions can be closed in the presence of high calcium concentrations. The effective concentration of Ca²⁺ can vary depending on cell type and type of connexin expressed.

In some aspects, channels can be opened and closed by changing the calcium concentration. For example, channels remained closed in the high calcium extracellular environment, trapping molecular cargo inside; however, upon forming junctions with cells, channels can open to release their cargo into the cytoplasm. In exemplary embodiments, liposomes are loaded with the chemotherapeutic doxorubicin and efficiently deliver their cargo to the cytoplasm of target cells, reducing the cytotoxic dose by more than an order of magnitude in comparison to extracellular delivery. Thus, gap junction liposomes of the present disclosure have the potential to substantially increase the efficiency with which existing drugs reach the cellular cytoplasm, as well as to enable the delivery of new drugs and reagents that are insoluble in the membrane environment. In addition, rapid delivery to cells can overcome chemotherapeutic resistance and overexpression of drug efflux pumps in response to stress.

The compound loaded into the vesicle can be any compound or composition that can be administered to animals, preferably humans. For example, the compound encompasses compositions that exert physiological activity in vivo and are effective for preventing or treating disease, for example, compounds or compositions used in diagnosis, such as contrast agents, and further encompasses genes useful for gene therapy. Examples of the physiologically active component can include preventive and therapeutic agents known in the art, such as calcium agents, active vitamin D3, calcitonin and derivatives thereof, peptides, β-alanyl-3,4-dihydroxyphenylalanine, xanthine derivatives, thrombomodulin, 17β-estradiol, steroid hormones, polyphenol compounds, prostaglandins, and interferon. Moreover, examples of the substance can include: central analgesics such as morphine, codeine, and pentazocine; steroid agents such as prednisolone, dexamethasone, and betamethasone; nonsteroidal anti-inflammatory agents such as aspirin, indomethacin, loxoprofen, and diclofenac sodium; and antiphlogistic analgesics such as antiphlogistic enzymes. Further examples of the compounds can include antirheumatic drugs such as sodium aurothiomalate, auranofin, D-penicillamine, bucillamine, lobenzarit, actarit, and salazosulfapyridine; immunosuppressive agents such as methotrexate, cyclophosphamide, azathioprine, and mizoribine; antiviral agents such as acyclovir, zidovudine, and interferons; antimicrobial agents such as aminoglycoside, cephalosporin, and tetracycline; polyene antibiotics; and antifungal agents such as imidazole and triazole. Examples of the substance can additionally include sterols (e.g., cholesterol), carbohydrate (e.g., sugar and starch), cell receptor proteins, immunoglobulin, enzymes, hormone, neurotransmitters, glycoproteins, peptides, proteins, dyes, radioactive labels (e.g., radioisotopes and radioisotope-labeled compounds), radiopaque compounds, fluorescent compounds, bronchodilators, and local anesthetics.

The vesicles according to the present disclosure preferably comprise antitumor agents. Examples of the antitumor agents include, but not particularly limited to, alkylating agents, antimetabolites of various types, antitumor antibiotics and other antitumor agents, antitumor plant components, BRM (biologically responsive modifier), antiangiogenic agents, cell adhesion inhibitors, matrix metalloprotease inhibitors, hormones and other chemotherapeutic drugs. Exemplary chemotherapeutics include doxorubicin, etoposide, paclitaxel, and gemcitabine.

The vesicles can comprise RNAi such as siRNA, shRNA, and miRNA. Manipulating the cellular process of RNA interference (RNAi) is an effective method for suppressing the expression of a specific gene to study its function. RNAi pathways are activated by various forms of double-stranded (ds) RNAs that contain sequences which are homologous to the mRNA transcript of a target gene. RNAi includes small interfering RNA (siRNA), short hairpin RNA (shRNA) and micro RNA (miRNA). Short hairpin RNA (shRNA) transcripts adopt a stable stem-loop structure in solution; can be easily be expressed from a cloned oligonucleotide template; and are a convenient and reproducible means of activating RNAi in cells. Small interfering RNA (siRNA) is a class of double-stranded RNA molecules about 20-25 nucleotides in length. siRNA interferes with the expression of specific genes with complementary nucleotide sequences by causing mRNA to be broken down after transcription, resulting in no translation.

C. Vesicle Aerosolization

The vesicles can be aerosolized for direct delivery to lung cells. Aerosols containing the vesicles can be generated by various methods known in the art (Gibbons and Smyth, 2011; incorporated herein by reference). For example, vesicles can be nebulized with a conventional air-jet nebulizer or a vibrating mesh nebulizer or aerosolized with metered-dose inhalers and dry powder inhalers. Vesicles can be formulated as either a suspension or as dry powder formulation. Powder formulation of the vesicles can be produced by various methods known in the art such as spray drying, or cryo methods such as thin film freezing method or lyophilization, using cryogenic stabilizers. A carrier, such as lactose, can be employed to disperse and deliver the dried vesicles to the lung. Preferably, the vesicles are aerosolized by a vibrating mesh nebulizer with vesicles suspended in an isotonic buffer. Alternatively, the aerosolization method can be the delivery of a dry powder formulation of vesicles by means of a dry powder inhaler. Usually, the dry formulation will contain particles with size between 1-5 μm, comprising both the carrier and attached vesicles.

D. Vesicle Targeting

Delivery of the vesicles to specific cells can be achieved by the addition of targeting ligands to the vesicles. Targeted vesicles have higher efficacy, protection from degradation and decreased drug toxicity. Further, by targeting receptors overexpressed on cancer cell surfaces (e.g., EGFR) with ligands expressed on the surfaces of vesicles, preferential interaction with tumor cells can be achieved. Exemplary ligands for use in the practice of the present disclosure include, but are not limited to, proteins (e.g., transferrin or folate), peptides (e.g., L-37pA), antibodies, antibody fragments (including Fab′ fragments and single chain Fv fragments (scFv)) and sugars (e.g., galactose), as well as other targeting molecules.

The targeting moiety can be any chemical composition that favors the positioning of a vesicle or liposome to a specific site or sites. More than one targeting moiety may be utilized on a single vesicle. The targeting moiety can be selected from the group consisting of a vitamin such as folate; transferrin; an antibody such as OVB-3, anti-CA125, anti-CEA, and others; sialyl Lewis X antigen, hyaluronic acid, mannose derivatives, glucose derivatives, cell specific lectins, galaptin, galectin, lactosylceramide, a steroid derivative, an RGD sequence, a ligand for a cell surface receptor such as epidermal growth factor (EGF), EGF-binding peptide, urokinase receptor binding peptide, a thrombospondin-derived peptide, an albumin derivative and/or a combinatorial molecule directed against various cells. In addition, tumor-homing peptides with vascular “zipcodes” (Teesalu et al., 2013) known in the art can be used for targeted the vesicles provided herein.

However, many targeting molecules such as antibodies and most protecting or stabilizing moieties such as polyethylene glycol (PEG) sterically inhibit the interaction between the liposomal membrane and the cell membrane, even though the liposome has bound to the cell surface. Because of this steric hindrance, it is generally not possible for fusogenic liposomes to efficiently deliver contents to the cytoplasm of the cell. Thus, targeted vesicles where at least a portion of the targeting/stabilizing moieties are at least temporarily removable at the target site such as U.S. Pat. No. 7,060,291, incorporate herein by reference, are also provided.

E. Vesicles Capable of Membrane Fusion

Vesicles may additionally comprise a fusogenic peptide embedded in the phospholipid membrane that provides the vesicle with the capability of interacting or fusing with a recipient cell membrane in a way that permits delivery of the transmembrane protein to the recipient cell membrane. Fusogenic peptides belong to a class of helical amphipathic peptides characterized by a hydrophobicity gradient along the long helical axis. This hydrophobicity gradient causes the tilted insertion of the peptides in membranes, thus destabilizing the lipid core and, thereby, enhancing membrane fusion (Decout et al., 1999).

The fusogenic peptides can be made by standard automated peptide synthesis. For example, the peptide is cleaved by standard techniques using trifluoroacetic acid (10 ml), water (0.5 ml), ethanedithiol (0.25 ml), and thioanisole (0.25 ml) (per gram of peptide-containing resin). The peptide (approximately 200 mg) is precipitated in 60 ml cold tert-butyl methyl ether, washed three times with cold tert-butyl methyl ether, redissolved in 10 ml of 1 mM hydrochloric acid, and lyophilized.

Alternatively, the fusogenic peptides are made by expression of nucleic acids encoding the fusogenic peptides. The nucleic acids can be naturally occurring and isolated, recombinately formed or chemically synthesized, i.e. by oligonucleotide synthesis. The amino acid sequences of the fusogenic peptides are provided and are generally relatively short, therefore, the codons encoding the desired sequence can be routinely selected (U.S. Pat. No. 6,372,720B1); incorporated herein by reference).

The vesicles provided herein, in combination with the fusogenic peptides and the substance(s) to be delivered (e.g., transmembrane protein), result in detectable liposome-cell fusion and delivery of the substance(s) contained therein and are effective in biological fluids including blood serum. For example, fusogenic peptides include TAT of HIV, hemagglutinin HA-2, HIV-1 transmembrane glycoprotein gp41, Alzheimer's bcia-amyloid peptide, and fusion peptide and N-terminal heptad repeat of Sendai virus (U.S. Pat. No. 6,511,676B1; incorporated herein by reference).

Hemagglutinin (HA) is a homotrimeric surface glycoprotein of the influenza virus. In infection, it induces membrane fusion between viral and endosomal membranes at low pH. Each monomer consists of the receptor-binding HA1 domain and the membrane-interacting HA2 domain. The NH2-terminal region of the HA2 domain (amino acids 1 to 127), the so-called “fusion peptide,” inserts into the target membrane and plays a crucial role in triggering fusion between the viral and endosomal membranes. Based on substitution of eight amino acids in the region 5-14 with cysteines and spin-labeling electron paramagnetic resonance it was concluded that the peptide forms an alpha-helix tilted approximately 25 degrees from the horizontal plane of the membrane with a maximum depth of 15 angstroms (A) from the phosphate group (Macosko et al., 1997). Use of fusogenic peptides from influenza virus hemagglutinin HA-2 enhanced greatly the efficiency of transferrin-polylysine-DNA complex uptake by cells; in this case the peptide was linked to polylysine and the complex was delivered by the transferrin receptor-mediated endocytosis (reviewed by Boulikas, 1998a). This peptide had the sequence: GLFEAIAGFIENGWEGMIDGGGYC (SEQ ID NO:1) and was able to induce the release of the fluorescent dye calcein from liposomes prepared with egg yolk phosphatidylcholine which was higher at acidic pH; this peptide was also able to increase up to 10-fold the anti-HIV potency of antisense oligonucleotides, at a concentration of 0.1-1 mM, using CEM-SS lymphocytes in culture. This peptide changes conformation at the slightly more acidic environment of the endosome destabilizing and breaking the endosomal membrane (reviewed by Boulikas, 1998a).

The presence of negatively charged lipids in the membrane is important for the manifestation of the fusogenic properties of some peptides but not of others; whereas the fusogenic action of a peptide, representing a putative fusion domain of fertilin, a sperm surface protein involved in sperm-egg fusion, was dependent upon the presence of negatively charged lipids. However, that of the HIV2 peptide was not (Martin and Ruysschaert, 1997).

For example, to analyze the two domains on the fusogenic peptides of influenza virus hemagglutinin HA, HA-chimeras were designed in which the cytoplasmic tail and/or transmembrane domain of HA was replaced with the corresponding domains of the fusogenic glycoprotein F of Sendai virus. Constructs of HA were made in which the cytoplasmic tail was replaced by peptides of human neurofibromin type 1 (NF1) (residues 1441 to 1518) or c-Raf-1, (residues 51 to 131). The constructs were expressed in CV-1 cells by using the vaccinia virus-T7 polymerase transient-expression system. Membrane fusion between CV-1 cells and bound human erythrocytes (RBCs) mediated by parental or chimeric HA proteins showed that, after the pH was lowered, a flow of the aqueous fluorophore calcein from preloaded RBCs into the cytoplasm of the protein-expressing CV-1 cells took place. This indicated that membrane fusion involves both leaflets of the lipid bilayers and leads to formation of an aqueous fusion pore (Schroth-Diez et al., 1998).

A remarkable discovery was that the TAT protein of HIV is able to cross cell membranes (Green and Loewenstein, 1988) and that a 36-amino acid domain of TAT, when chemically crosslinked to heterologous proteins, conferred the ability to transduce into cells. It is worth mentioning that the 11-amino acid fusogenic peptide of TAT (YGRKKRRQRRR (SEQ ID NO:2)) is a nucleolar localization signal (see Boulikas, 1998b).

Another protein of HIV, the glycoprotein gp41, contains fusogenic peptides. Linear peptides derived from the membrane proximal region of the gp41 ectodomain have potential applications as anti-HIV agents and inhibit infectivity by adopting a helical conformation (Judice et al., 1997). The 23 amino acid residues N-terminal peptide of HIV-1 gp41 has the capacity to destabilize negatively charged large unilamellar vesicles. In the absence of cations the main structure was a pore-forming alpha-helix, whereas in the presence of Ca2⁺ the conformation switched to a fusogenic, predominantly extended beta-type structure. The fusion activity of HIV(ala) (bearing the R22(A substitution) was reduced by 70% whereas fusogenicity was completely abolished when a second substitution (V2(E) was included arguing that it is not an alpha-helical but an extended structure adopted by the HIV-1 fusion peptide that actively destabilizes cholesterol-containing, electrically neutral membranes (Pereira et al., 1997).

The C-terminal fragments of the Alzheimer amyloid peptide (amino acids 29-40 and 29-42) have properties related to those of the fusion peptides of viral proteins inducing fusion of liposomes in vitro. These properties could mediate a direct interaction of the amyloid peptide with cell membranes and account for part of the cytotoxicity of the amyloid peptide. In view of the epidemiologic and biochemical linkages between the pathology of Alzheimer's disease and apolipoprotein E (apoE) polymorphism, examination of the potential interaction between the three common apoE isoforms and the C-terminal fragments of the amyloid peptide showed that only apoE2 and apoE3, not apoE4, are potent inhibitors of the amyloid peptide fusogenic and aggregational properties. The protective effect of apoE against the formation of amyloid aggregates was thought to be mediated by the formation of stable apoE/amyloid peptide complexes (Pillot et al., 1997a; Lins et al., 1999).

The fusogenic properties of an amphipathic net-negative peptide (WAE 11), consisting of 11 amino acid residues were strongly promoted when the peptide was anchored to a liposomal membrane; the fusion activity of the peptide appeared to be independent of pH and membrane merging and the target membranes required a positive charge which was provided by incorporating lysine-coupled phosphatidylethanolamine (PE-K). Whereas the coupled peptide could cause vesicle aggregation via nonspecific electrostatic interaction with PE-K, the free peptide failed to induce aggregation of PE-K vesicles (Pecheur et al., 1997).

Alternatively, addition of a small amount of cationic lipids replacing positive charges of the vesicle also endows the vesicle with fusogenic properties. The percentage of positive charges to be substituted by cationic lipids is small because of the toxicity of cationic lipids. For example, cationic lipids include DDAB, dimethyldioctadecyl ammonium bromide; DMRIE: N-[1-(2,3-dimyristyloxy)propyl]-N,N-dimethyl-N-(2-hydroxyethyl) ammonium bromide; DMTAP: 1,2-dimyristoyl-3-trimethylammonium propane; DOGS: Dioctadecylamidoglycylspermine; DOTAP: N-(1-(2,3-dioleoyloxy)propyl)-N,N,N-trimethylammonium chloride; DPTAP: 1,2-dipalmitoyl-3-trimethylammonium propane; and DSTAP: 1,2-disteroyl-3-trimethylammonium propane.

### IV. METHODS OF TREATMENT

A. Restoring Gap Junction Network

Development and maintenance of healthy tissues requires that cells communicate with their neighbors. Poor intercellular communication plays a role in the initiation and progression of a remarkable number of diseases from growth and metastasis of cancerous tumors to chronic inflammation in asthma and atherosclerosis (Naus et al., 2010). Presently, no therapeutic options exist to directly address the loss of intercellular communication in diseased tissues. Incorporating intracellular junctions in vesicles is challenging because junctions consist of transmembrane proteins. These complex molecules are notoriously difficult to isolate and manipulate.

Methods of treating a disease or condition in a subject in need thereof are provided herein comprising administering to the patient an effective amount of a therapeutic transmembrane protein such as by administering plasma membrane vesicles. In certain embodiments, the vesicles are for the treatment of a disease such as cancer in which functional gap junctions are serve to enhance or restore the gap junction network through the delivery of vesicles with functional gap junctions. The administration of the vesicles can integrate the gap junctions into the membranes of the cancer cells. Alternatively, the vesicles can serve as bridges between cancer cells connected through the vesicle gap junctions.

Vesicles with a high concentration of functional connexin pores are extracted directly from the plasma membrane of healthy donor cells by the methods described herein. Vesicles with gap junctions can integrate into the remaining gap junctions among cancer cells, helping to rebuild the junction network and simultaneously opening efficient passageways for the delivery of drugs to the cellular cytoplasm. Robust gap junction networks are well known to suppress tumor growth and invasion by promoting tissue homeostasis and transmission of biochemical signals from neighboring healthy tissues.

Certain aspects of the methods disclosed herein use the cellular gap junction network to deliver drugs directly to the cytoplasm of tumor cells. Channels can form when connexin pores on the surfaces of two cells come together, enabling cells to share metabolites, second messengers, peptides and microRNAs. In particular embodiments, vesicles with gap junctions can restore gap junctional communication and deliver a compound such as chemotherapeutic drugs. In a process known as the bystander effect, chemotherapeutics spread from cell to cell using the gap junction network, enhancing penetration of drugs within tumors. The therapeutic vesicles provided herein can actively participate in the gap junction network and drugs encapsulated within the particles can be delivered directly to the cytoplasm of tumor cells via transmembrane channels, bypassing the inefficient endocytic route.

B. Restoring Transmembrane Protein Function

In some embodiments, methods are provided for the treatment of a disease or condition in a patient in need thereof comprising the administration of vesicles with functional transmembrane proteins. In one embodiment, a patient with cystic fibrosis is treated by the administration of vesicles with functional, wild-type cystic fibrosis transmembrane conductance regulator (CFTR). The wild-type CFTR can be delivered to cells by the vesicles provided herein in an effective amount formulated as an aerosol for inhalation directly to lung cells.

There are numerous diseases with mutated or dysfunctional transmembrane proteins that can be treated by the methods disclosed herein such as cancer, cystic fibrosis, CNS diseases, metabolic disorders, asthma, atherosclerosis and orphan diseases. For example, other transmembrane proteins that can be delivered to cells by the methods provided herein to restore function and for the treatment of diseases include, but are not limited to, thyrotopin receptor or TSH receptor mutated in congenital hyperthyroidism; myelin protein zero mutations associated with Charcot-Marie-Tooth disease and Dejerine-Sottas disease; Connexin 26 mutation associated with hereditary deafness; Melacortin 4 receptor mutated in inherited obesity; Receptor tyrosine-protein kinase erbB-2 mutated in breast cancer; Myelin proteolipid protein mutated in Pelizaeus-Merzbacher disease; Low-density lipoprotein mutated in familial hypercholesterolemia; Beta amyloid peptide misfolding in Alzheimer's disease; and gap junction proteins involved in skin abnormalities. Skin diseases with mutated Connexin 26 include Vohwinkel syndrome (VS), keratitis-ichthyosis-deafness (KID) syndrome, Bart-Pumphrey syndrome (BPS) and hystrix-like ichthyosis-deafness (HID) syndrome.

In some embodiments, the cancer being treated is, but is not limited to, a primary or metastatic brain tumor, neuroendocrine tumors, melanoma, prostate, head and neck, ovarian, lung, kidney, liver, breast, vaginal, urogenital, gastric, colorectal, cervical, liposarcoma, angiosarcoma, rhabdomyosarcoma, choriocarcinoma, pancreatic, retinoblastoma, multiple myeloma and other types of cancer. In particular embodiments, the cancer is metastatic breast cancer.

1. Pharmaceutical Formulation

Also provided herein is a pharmaceutical composition comprising a pharmaceutically acceptable carrier and the vesicles disclosed herein. Said composition is useful, for example, in the delivery of transmembrane proteins to the cells of an animal. Pharmaceutically acceptable carriers are generally formulated according to a number of factors well within the purview of the ordinarily skilled artisan to determine and account for, including without limitation: the particular liposomal bioactive agent used, its concentration, stability and intended bioavailability; the disease, disorder or condition being treated with the liposomal composition; the subject, its age, size and general condition; and the composition's intended route of administration, e.g., nasal, oral, ophthalmic, topical, transdermal, vaginal, subcutaneous, intramammary, intraperitoneal, intravenous, or intramuscular (see, for example, Nairn (1985), the contents of which are incorporated herein by reference). Typical pharmaceutically acceptable carriers used in parenteral bioactive agent administration include, for example, D5W, an aqueous solution containing 5% weight by volume of dextrose, and physiological saline. Pharmaceutically acceptable carriers can contain additional ingredients, for example those which enhance the stability of the active ingredients included, such as preservatives and anti-oxidants.

The pharmaceutical excipient may be a liquid or solid filler, diluent, solvent or encapsulating material, involved in carrying or transporting any subject composition or component thereof from one organ, or portion of the body, to another organ, or portion of the body. Each excipient must be “acceptable” in the sense of being compatible with the subject composition and its components and not injurious to the patient. Suitable excipients include trehalose, raffinose, mannitol, sucrose, leucine, trileucine, and calcium chloride. Examples of other suitable excipients include (1) sugars, such as lactose, and glucose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, coconut oil, avocado oil, and soybean oil; (10) glycols, such as propylene glycol; (11) polyols, such as glycerin, sorbitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The methods of the present disclosure can further comprise administering an additional therapy to the patient in combination with the vesicles provided herein. The additional therapy can comprise the administration of a chemotherapeutic agent, a small molecule, radiation therapy or nucleic acid based therapy.

2. Dosages

The dosage of any compositions of the present disclosure will vary depending on the symptoms, age and body weight of the patient, the nature and severity of the disorder to be treated or prevented, the route of administration, and the form of the subject composition. Any of the subject formulations may be administered in a single dose or in divided doses. Dosages for the compositions of the present disclosure may be readily determined by techniques known to those of skill in the art or as taught herein.

In certain embodiments, the dosage of the subject compounds will generally be in the range of about 0.01 ng to about 10 g per kg body weight, specifically in the range of about 1 ng to about 0.1 g per kg, and more specifically in the range of about 100 ng to about 10 mg per kg.

An effective dose or amount, and any possible effects on the timing of administration of the formulation, may need to be identified for any particular composition of the present disclosure. This may be accomplished by routine experiment as described herein, using one or more groups of animals (preferably at least 5 animals per group), or in human trials if appropriate. The effectiveness of any subject composition and method of treatment or prevention may be assessed by administering the composition and assessing the effect of the administration by measuring one or more applicable indices, and comparing the post-treatment values of these indices to the values of the same indices prior to treatment.

The precise time of administration and amount of any particular subject composition that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

3. Inhalation Device

The pharmaceutical formulations of the present disclosure may be used in any dosage dispensing device adapted for intranasal administration. The device should be constructed with a view to ascertaining optimum metering accuracy and compatibility of its constructive elements, such as container, valve and actuator with the nasal formulation and could be based on a mechanical pump system, e.g., that of a metered-dose nebulizer, dry powder inhaler, soft mist inhaler, or a nebulizer. Due to the large administered dose, preferred devices include jet nebulizers (e.g., PARI LC Star, AKITA), soft mist inhalers (e.g., PARI e-Flow), and capsule-based dry powder inhalers (e.g., PH&T Turbospin). Suitable propellants may be selected among such gases as fluorocarbons, hydrocarbons, nitrogen and dinitrogen oxide or mixtures thereof.

The inhalation delivery device can be a nebulizer or a metered dose inhaler (MDI), or any other suitable inhalation delivery device known to one of ordinary skill in the art. The device can contain and be used to deliver a single dose of the antiinfective compositions or the device can contain and be used to deliver multi-doses of the compositions of the present disclosure.

A nebulizer type inhalation delivery device can contain the compositions of the present disclosure as a solution, usually aqueous, or a suspension. In generating the nebulized spray of the compositions for inhalation, the nebulizer type delivery device may be driven ultrasonically, by compressed air, by other gases, electronically or mechanically. The ultrasonic nebulizer device usually works by imposing a rapidly oscillating waveform onto the liquid film of the formulation via an electrochemical vibrating surface. At a given amplitude the waveform becomes unstable, whereby it disintegrates the liquids film, and it produces small droplets of the formulation. The nebulizer device driven by air or other gases operates on the basis that a high pressure gas stream produces a local pressure drop that draws the liquid formulation into the stream of gases via capillary action. This fine liquid stream is then disintegrated by shear forces. The nebulizer may be portable and hand held in design, and may be equipped with a self-contained electrical unit. The nebulizer device may comprise a nozzle that has two coincident outlet channels of defined aperture size through which the liquid formulation can be accelerated. This results in impaction of the two streams and atomization of the formulation. The nebulizer may use a mechanical actuator to force the liquid formulation through a multiorifice nozzle of defined aperture size(s) to produce an aerosol of the formulation for inhalation. In the design of single dose nebulizers, blister packs containing single doses of the formulation may be employed.

In the present disclosure the nebulizer may be employed to ensure the sizing of particles is optimal for positioning of the particle within, for example, the pulmonary membrane.

A metered dose inhalator (MDI) may be employed as the inhalation delivery device for the compositions of the present disclosure. This device is pressurized (pMDI) and its basic structure comprises a metering valve, an actuator and a container. A propellant is used to discharge the formulation from the device. The composition may consist of particles of a defined size suspended in the pressurized propellant(s) liquid, or the composition can be in a solution or suspension of pressurized liquid propellant(s). The propellants used are primarily atmospheric friendly hydroflourocarbons (HFCs) such as 134a and 227. Traditional chloroflourocarbons like CFC-11, 12 and 114 are used only when essential. The device of the inhalation system may deliver a single dose via, e.g., a blister pack, or it may be multi dose in design. The pressurized metered dose inhalator of the inhalation system can be breath actuated to deliver an accurate dose of the lipid-containing formulation. To insure accuracy of dosing, the delivery of the formulation may be programmed via a microprocessor to occur at a certain point in the inhalation cycle. The MDI may be portable and hand held.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Gap Junction Vesicle Formation

Cells use the gap junction network to share small molecules, including metabolites, drugs, and small interfering RNAs, directly with each other. The proteins that form gap junctions are called connexins, and they assemble into hexameric hemichannels called connexons, in the plasma membranes of cells (Andrade-Rozental et al., 2000). When connexon hemichannels within the plasma membranes of two neighboring cells meet, they form a complete gap junction channel that connects the two cells, enabling molecules in the cytoplasm of one cell to diffuse through the channel and into the cytoplasm of a neighboring cell. Gap junctions transfer chemotherapeutics from cell to cell, enabling drug penetration in tumors (Yamasaki et al., 1999). This phenomenon, known as the bystander effect (Fujimoto et al., 1971) promotes the efficacy of a diverse range of chemotherapeutics such as doxorubicin, etoposide, paclitaxel, gemcitabine, and others (Huang et al., 2001). Inspired by the potential utility of gap junctions for drug delivery, cargo-loaded liposomes that can form functional gap junctions with cells were created. Direct release of molecular cargo into the cytoplasm is demonstrated by this approach, reducing the effective concentration of the model chemotherapeutic, doxorubicin, by an order of magnitude.

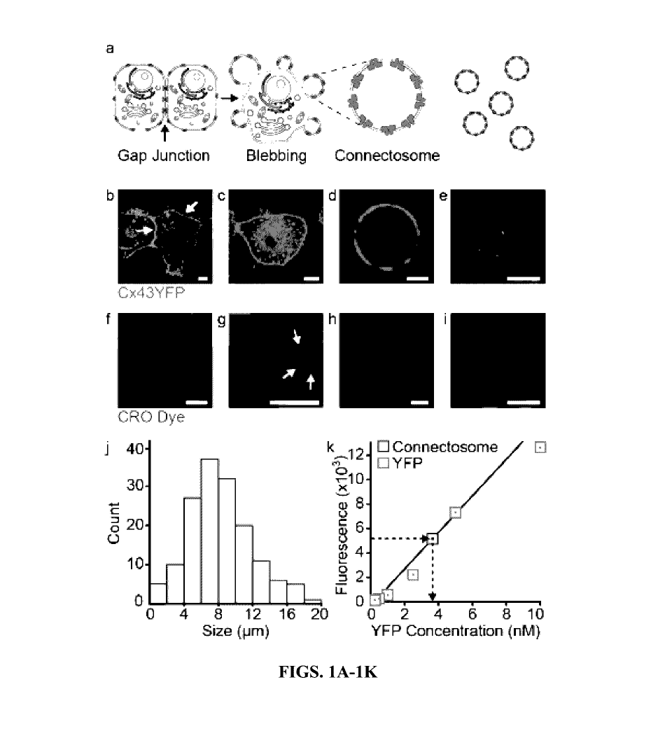

Giant plasma membrane vesicles (GPMVs) were directly harvested from the plasma membranes of donor cells that overexpress connexin proteins and contain a high density of functional, properly oriented hemichannels directly from the plasma membrane of mammalian donor cells through a process known as zeiosis, or plasma membrane blebbing (FIG. 1A). Membrane blebbing is involved in multiple physiological processes including cell motility (Charras 2008), mitosis (Cunningham 1995; Laster 1996; Boucrot 2007), chemotaxis (Blaser 2006), viral entry (Mercer and Helenius 2008), and maintenance of cell polarity (Ohta 2006), morphology (Vidali 2006), and mechanical homeostasis (Aranda-Espinoza 2010). Plasma membrane blebs form when attachments between the plasma membrane and the cytoskeleton are disrupted (Mahadevan 2008). By extracting blebs from donor cells that overexpressed connexin 43 proteins with a C-terminal YFP modification (Cx43-YFP), Connectosomes with embedded connexin 43-YFP were developed (FIG. 1A-E). Over 90% of these Connectosomes contained connexin 43-YFP at levels detectable by fluorescence imaging. The Connectosomes ranged in diameter from 4 to more than 20 μm, with an average diameter of 10 μm (FIG. 1J). Notably, Connectosomes can be extruded to reduce their diameter to around 100 nanometers. Based on quantitative measurements of YFP fluorescence, it was determined that the average Connectosome contained over 400,000 connexons, which cumulatively covered nearly 10% of the vesicle surface (FIG. 1K).

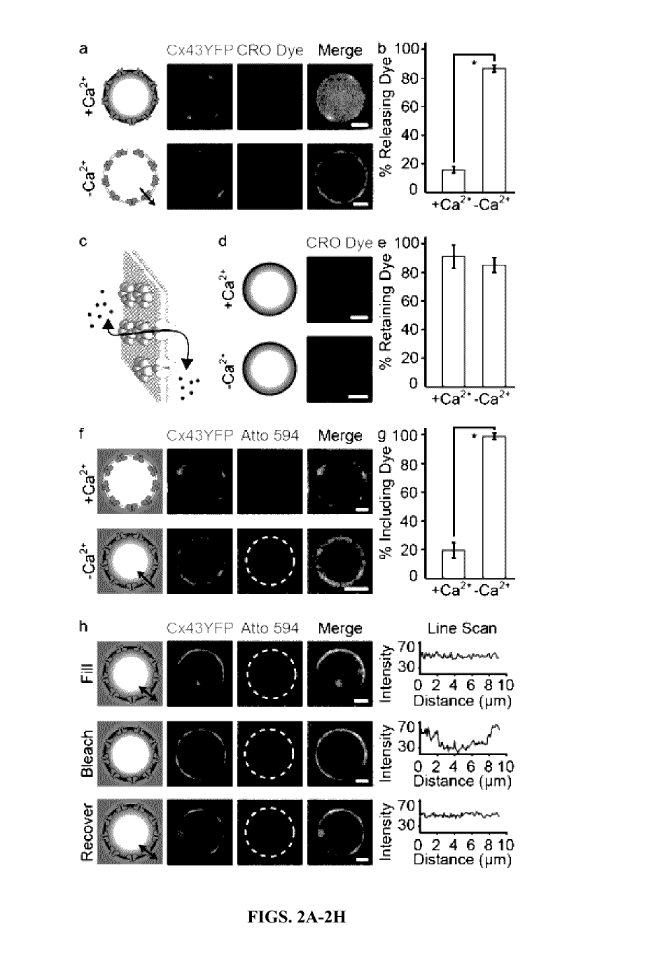

To test the functionality of connexon channels embedded in Connectosomes, the ability of the channels to open and close was examined in the absence and presence of calcium. Specifically, it is well established that calcium causes unpaired connexons to close, obstructing the passage of molecules (Thimm et al., 2005; Allen et al., 2011; Muller et al., 2002). However, in the absence of calcium, connexons undergo a conformational change that causes them to open, allowing small molecules to diffuse through them (Allen et al., 2011). The ability of connexons to open upon calcium removal, releasing dye encapsulated within the Connectosomes, was examined. To load Connectosomes with the dye the donor cells were treated with calcein red-orange (CRO) acetomethoxy (AM) prior to extracting membrane blebs (FIG. 1F-I). CRO AM diffuses freely across the plasma membrane. However, when the dye reaches the cytoplasm, intracellular esterases hydrolyze the acetomethoxy group. The resulting CRO dye molecule is membrane impermeable, trapped inside of the cell and permeable only to gap junctions (FIG. 1F) (Al-Mehdi 2008). In the presence of calcium, the Connectosomes retained the CRO dye (FIG. 2A, top). However, when calcium was removed by addition of EGTA and EDTA chelators, the dye was released from 87% of the Connectosomes and retained by only 13%, demonstrating that the connexons opened (FIG. 2A, bottom; B-C).

To further illustrate the dependence of dye release on the presence of functional connexons in the Connectosomes, CRO dye-loaded plasma membrane blebs were formed from MDA-MB-231 donor cells. MDA-MB-231 cells express low levels of connexin 43 and exhibit defective connexin trafficking and gap junction formation, resulting in substantially reduced gap junction intercellular communication. In the presence of calcium, 91% of MDA-MB-231 blebs retained the dye (FIG. 2D, top). When calcium was removed by addition of EGTA and EDTA chelators, 85% of the MDA-MB-231 blebs continued to retain the dye, in comparison to only 13% of Connectosomes, demonstrating that the dye release was dependent on the presence of functional connexons (FIG. 2D, bottom; E).

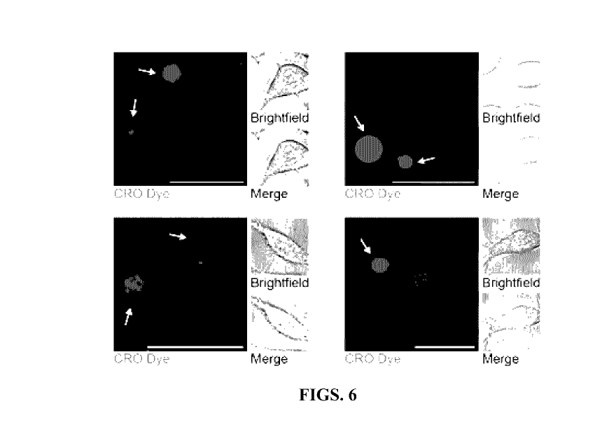

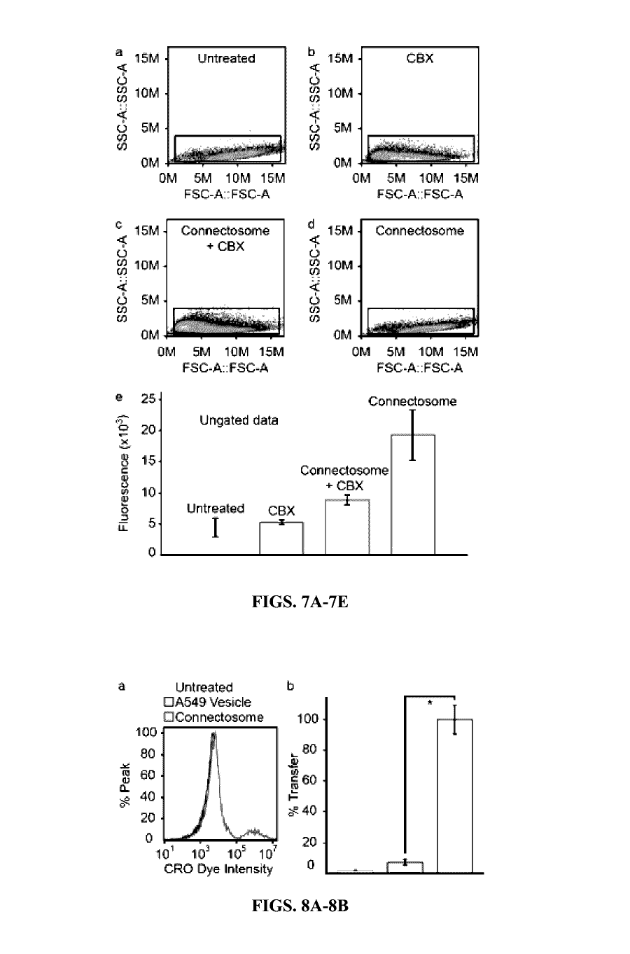

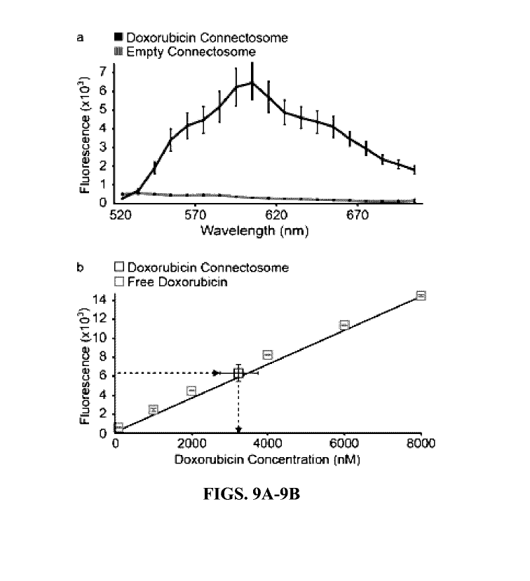

Next, an exogenous method of loading was developed, in which molecular cargo was encapsulated after Connectosome formation. Specifically, a water-soluble dye with little or no membrane permeability, Atto 594, was added to the solution surrounding pre-formed Connectosomes. In the presence of calcium, the Connectosomes excluded the dye, demonstrating that connexons remained closed (FIG. 2F, top). However, when calcium was removed by addition of EGTA and EDTA chelators, 99% of the Connectosomes filled with dye, demonstrating that the connexons opened (FIG. 2F, bottom; G). Similar results were also obtained for Connectosomes loaded with Atto 488 dye using an identical protocol (FIG. 5). Atto 488 has been reported to have no significant interaction with membranes, making the dye almost perfectly membrane impermeable.

Finally, to probe the timescale of diffusion through open connexons, photobleached Atto 594 dye was loaded within the Connectosomes. In the absence of calcium, the Connectosomes refilled with dye within 75 seconds after photobleaching (FIG. 2H). Together, these results demonstrate two distinct modes of loading Connectosomes and demonstrate that Connectosomes contain multiple functional connexons, capable of opening and closing to enable rapid molecular exchange with the external environment. Further, comparison to MDA-MB-231 blebs suggests that molecular exchange is connexon-dependent.

### Example 2—Drug Delivery by Gap Junction Vesicles

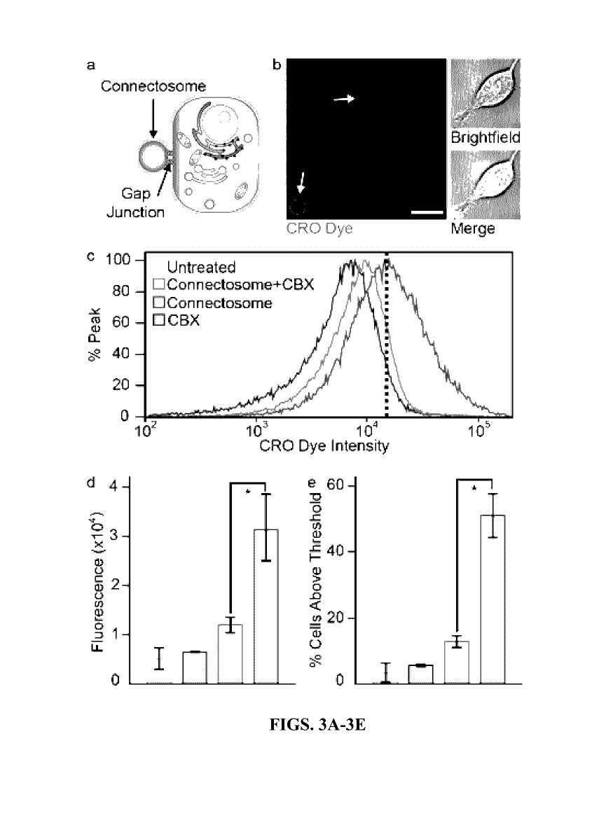

Having established the functionality of the connexons, the ability of the Connectosomes to deliver molecular cargo into the cellular cytoplasm was examined (FIG. 3A). While the presence of calcium keeps unpaired connexons closed (Allen et al., 2011), complete channels form and open when two unpaired connexons on the surfaces of neighboring cells meet, even in the presence of physiological levels of extracellular calicium (Sakhtianchi et al., 2013). To test the ability of Connectosomes to form gap junctions with cells, a confluent monolayer of recipient HeLa cells was prepared. CRO dye-loaded Connectosomes were prepared as described above (FIG. 1F-I) and incubated with the recipient cells. Imaging the recipient cells after 2 hours revealed the intracellular accumulation of dye (FIG. 3B, 6). To quantify the CRO dye delivery, the relative fluorescence intensity of the cell populations was measured using flow cytometry (FIG. 3C-E). Exposure to CRO dye-loaded Connectosomes increased the average fluorescence of the recipient cells by a factor of 6, in comparison to background fluorescence from untreated cells (FIG. 3D, 6). Additionally, a threshold was drawn at the peak of the fluorescence histogram for cells receiving dye-loaded Connectosomes (FIG. 3C). The average percentage of cells with fluorescence greater than this threshold increased from less than 4% for untreated cells to over 51% for cells exposed to dye-loaded Connectosomes (FIG. 3E). To demonstrate that the CRO dye delivery was gap junction-dependent, carbenoxolone (Al-Ghamdi, 2008) (CBX), a drug which blocks the coupling of connexons, to inhibit the formation of gap junctions between Connectosomes and recipient HeLa cells. Repeating the dye delivery experiment in the presence of this gap junction inhibitor significantly decreased the average recipient cell fluorescence, illustrating that dye delivery was dependent on the assembly of gap junction channels between the Connectosomes and the cells (FIG. 3C-E, 6). CBX treatment did not completely eliminate the increase in fluorescence of the recipient cells upon exposure to dye-loaded Connectosomes, likely because CBX is not a complete inhibitor of gap junction communication (Connors, 2012) and because CBX itself somewhat increases the fluorescence of the recipient cells, in the absence of Connectosome treatment (FIG. 3C-E, 7).

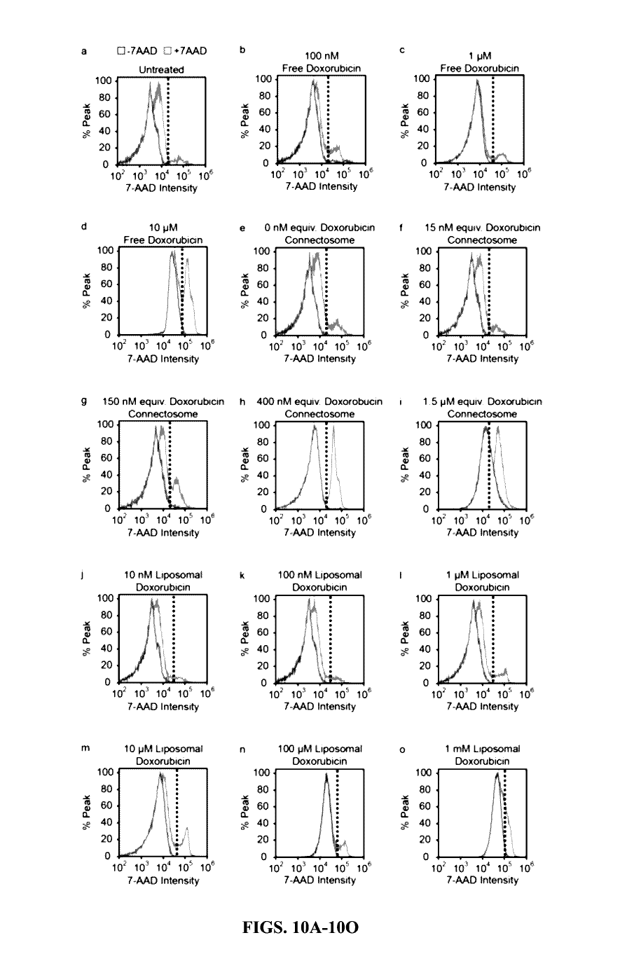

To further demonstrate the gap junction-dependence of the CRO dye delivery, the same experiment as above was repeated, using plasma membrane vesicles that lacked a significant concentration of functional connexons (FIG. 8). Specifically, CRO dye-loaded plasma membrane vesicles were formed from A549 cells, which are known to have low levels of connexin expression and gap junctional communication (Connors, 2012). These connexon-lacking plasma membrane vesicles were incubated with recipient HeLa cells, and the relative recipient cell fluorescence was measured using flow cytometry (FIG. 8). It was found that the fluorescence signal from cells exposed to CRO dye-loaded A549 vesicles was more than an order of magnitude less than the average fluorescence signal from cells exposed to Connectosomes. Collectively, these results demonstrate gap junction-dependent delivery of molecular cargo using Connectosomes.

Next, the use of Connectosomes to deliver the chemotherapeutic doxorubicin to the cellular cytoplasm was investigated. Doxorubicin was used because its inherent fluorescence allowed visualization of its encapsulation within Connectosomes. It was noted that doxorubicin may not be an ideal candidate for delivery via Connectosomes, owing to its cardiotoxicity and the importance of connexins in heart tissue. However, any small molecule drug or biomolecule can in principle be encapsulated within Connectosomes, and nanoparticles in general have not been observed to accumulate in the heart. Further, incorporation of targeting ligands has recently been demonstrated to dramatically increase binding specificity of cell derived vesicles to target cells overexpressing biomarkers such as the epidermal growth factor receptor (EGFR).

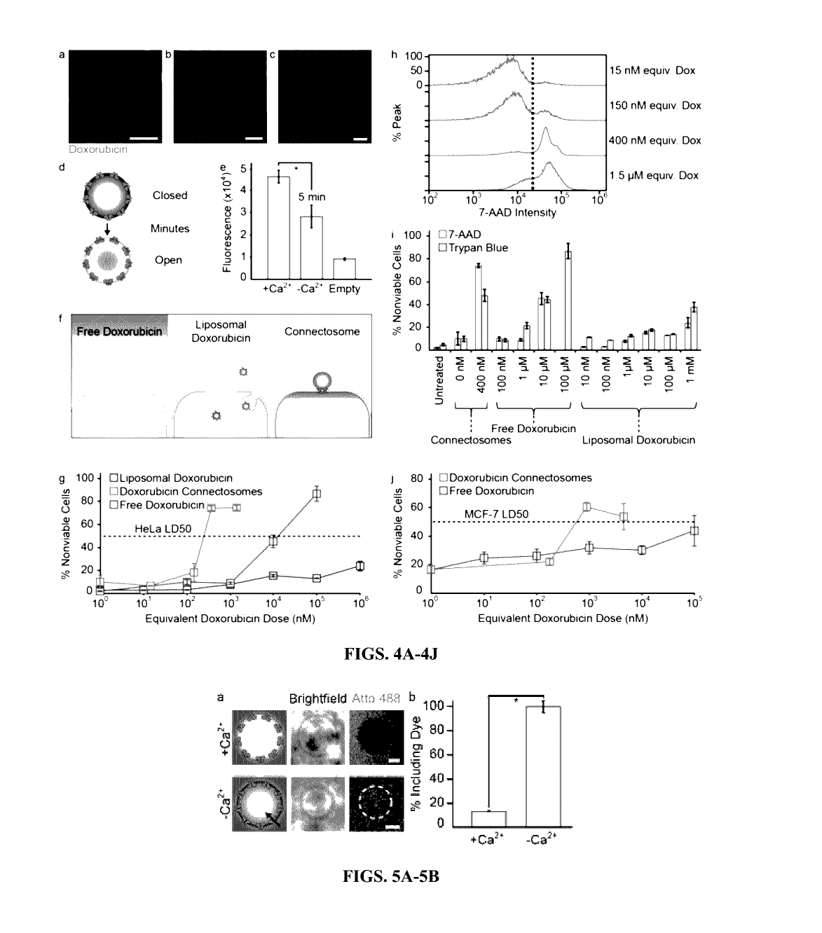

To encapsulate doxorubicin within Connectosomes, donor cells were treated with doxorubicin (FIG. 4A), such that the blebs derived from these cells contained the drug (FIG. 4B-C). Notably, chemotherapeutics such as doxorubicin require 2-3 days to substantially impact cell viability, while harvesting Connectosomes requires only a few hours. Therefore, loss of donor cell viability owing to drug loading was found to be insignificant during the Connectosome production process. Additionally, it is important to note that doxorubicin could be encapsulated within Connectosomes either by loading the cells with the semi-membrane permeable drug, or by opening and subsequently closing the connexons of preformed Connectosomes in the presence of a solution of the drug. Loading of the cells prior to Connectosome extraction was found to slightly increase the concentration of encapsulated drug and the overall material yield (i.e., Connectosomes per donor cell) and was therefore used to produce the Connectosomes for the doxorubicin studies presented here.

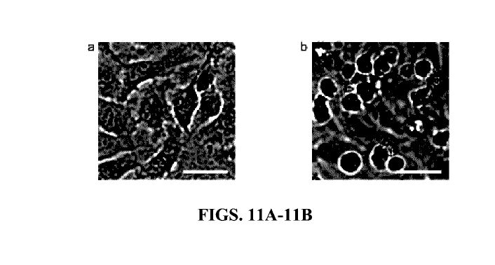

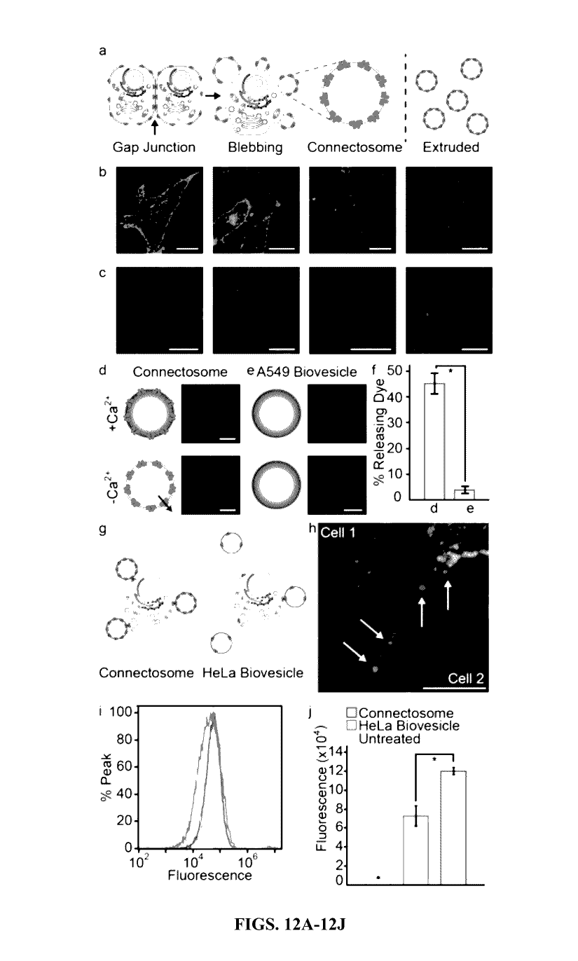

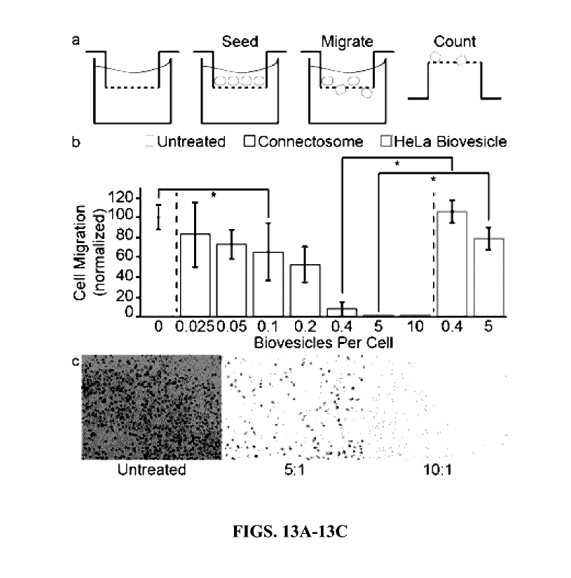

The doxorubicin content of the Connectosomes was quantified by measuring their fluorescence emission after resuspending them in fresh solution (FIG. 9). The native fluorescence of empty Connectosomes was measured and determined negligible. Based on the peak fluorescence emission of each Connectosome sample and a calibration curve of free doxorubicin fluorescence emission, we were able to determine that the average concentration of doxorubicin in each Connectosome sample was in the micromolar range. Based on this value as well as the average diameter and number of Connectosomes per volume, it was estimated that the concentration of doxorubicin within Connectosomes was approximately 1 mM. Notably, this concentration could be further increased by crystallizing doxorubicin within vesicles, as is done in the preparation of conventional liposomal formulations.

It was then investigated the timescale of doxorubicin release from Connectosomes (FIG. 4D). To begin, the fluorescence of doxorubicin-loaded Connectosomes was measured using flow cytometry (FIG. 4E). After addition of EGTA and EDTA chelators to remove residual calcium and open connexons, the average doxorubicin fluorescence of the Connectosomes decreased significantly within 5 minutes. These results demonstrate the potential for rapid drug release upon connexon opening. In contrast, when chelators were not added, the vesicles retained their content throughout the time course of all experiments.

Next, a control study was conducted in which the viability of a confluent monolayer of HeLa cells was measured 24 hours after free doxorubicin was added directly to the cell media at increasing concentrations from 100 nM to 100 μM (FIG. 4F-G, I, 10). The cytotoxic dose of doxorubicin for HeLa cells after 24 hours of exposure is approximately 10 μM (Al-Ghamdi, 2008). Cell viability was evaluated using both trypan blue and 7-AAD cell permeability assays on at least 3 independent populations of cells per condition per stain. As expected, a trend of decreasing cell viability was found with increasing doxorubicin concentration. Specifically, while a dose of 100 nM was not significantly cytotoxic (9% trypan blue/10% 7-AAD), the percentage of nonviable cells increased with increasing doxorubicin dose at 1 μM (21% trypan blue/9% 7-AAD), 10 μM (44% trypan blue/45% 7-AAD), and 100 μM (87% trypan blue) (FIG. 4G, I). Cells receiving 100 μM doxorubicin were outside the range of sensitivity for the 7-AAD assay, therefore, the percentage of nonviable cells at this concentration measured using the trypan blue assay was used in FIG. 4G.

Then, a study was conducted in which the viability of a confluent monolayer of HeLa cells was measured 24 hours after conventional, commercially sourced liposomal doxorubicin was added directly to the cell media at increasing doxorubicin concentrations from 10 nM to 1 mM (FIG. 4F-G, I, 10). These experimental parameters are consistent with the systemic infusions used to administer liposomal doxorubicin in the clinical setting. Cell viability was evaluated using both trypan blue and 7-AAD cell permeability assays on at least 3 independent populations of cells per condition per stain. It was found that the LD50 of liposomal doxorubicin was more than an order of magnitude greater than the LD50 of free doxorubicin (FIG. 4G, I).

Finally, confluent HeLa cell monolayers were exposed to doxorubicin-loaded Connectosomes for 2 hours (FIG. 4F). Independent cell samples were exposed to increasing concentrations of Connectosomes, which were equivalent in terms of total doxorubicin content to free doxorubicin concentrations of 15 nM, 150 nM, 400 nM, and 1.5 μM. As discussed above, these concentrations were determined by measuring the doxorubicin fluorescence emission for each sample (FIG. 9). While the 15 nM Connectosome dose was not significantly cytotoxic (7% 7-AAD), the percentage of nonviable cells increased with increasing Connectosome concentration at 150 nM (18% 7-AAD), 400 nM (74% 7-AAD), and 1.5 μM (75% 7-AAD) (FIG. 4g-h, Supporting Information FIG. S7). To confirm these results, the experiment was repeated with doxorubicin-loaded Connectosomes at the lowest effective dose, 400 nM, measuring viability using the trypan blue assay (FIG. 41). The results of this study were comparable to the results of the 7-AAD assay.

To test Connectosomes in a second model cell line, the assay was repeated using recipient MCF-7 cells. MCF-7 cells are human breast adenocarcinoma cells that have been used frequently in studies of drug delivery materials. First, a control study was conducted in which the viability of a confluent monolayer of MCF-7 cells was measured 48 hours after free doxorubicin was added directly to the cell media at increasing concentrations from 10 nM to 100 μM (FIG. 4J). Next, independent cell samples were exposed to increasing concentrations of Connectosomes, which were equivalent in terms of total doxorubicin content to free doxorubicin concentrations of 180 nM, 900 nM, and 4.5 μM. While the majority of cells treated with Connectosomes at an equivalent doxorubicin concentration of 900 nM were nonviable (61% 7-AAD trypan blue), the majority of cells treated with free doxorubicin remained viable even at a concentration of 100 μM (44% trypan blue) (FIG. 4J).

As illustrated by these collective results, the therapeutically effective dose (LD50) of doxorubicin increases by more than an order of magnitude when the drug is encapsulated within a conventional liposome, rather than administered to cells as a free drug in solution. This result, which is in agreement with the original literature on liposomal doxorubicin in vitro, points to a key limitation of liposomal formulations that has prevented their broad clinical adoption to date. Specifically, their ability to concentrate drugs is largely negated by a corresponding reduction in the availability of the encapsulated drug to the cellular cytoplasm. In contrast, the LD50 for doxorubicin-loaded Connectosomes is more than an order of magnitude less than the LD50 for free doxorubicin and several orders of magnitude less than the LD50 for liposomal doxorubicin. These results illustrate the ability of Connectosomes to dramatically increase the efficiency of drug delivery to the cellular cytoplasm, removing a key limitation of liposomal formulations.

### Example 3—Materials and Methods

Reagents.

CellTrace Calcein Red-Orange AM and trypan blue were purchased from Life Technologies. Sodium phosphate, DTT (dithiothreitol), PFA (paraformaldehyde), doxycycline, glycine, Atto 594-NHS ester, imidazole, NaCl, CaCl₂, EGTA (ethylene glycol tetraacetic acid), EDTA (ethylenediaminetetraacetic acid), HEPES (4-(2-hydroxyethyl)-1-piperazineethanesulfonic acid), DMSO (dimethyl sulfoxide) and doxorubicin were purchased from Sigma. Fetal bovine serum (FBS), trypsin, penicillin, streptomycin, L-glutamine, PBS (phosphate buffered saline), and DMEM (Dulbecco's modified Eagle medium) were purchased from GE Healthcare. Puromyocin was purchased from Clontech. Geneticin (G418) was purchased from Corning. Leupeptin and pepstatin were purchased from Roche. PMSF (phenylmethanesulfonyl fluoride), β-ME (β-mercaptoethanol) were purchased from Fisher Scientific. 7-AAD (7-amino-actinomycin D) was purchased from Affymetrix eBioscience. All chemical reagents were used without further purification.

Cell Culture.

Stably transfected, inducible tet-on cells expressing connexin 43 with a C terminal YFP modification were a gift from Matthias Falk (Fong et al., 2012; Lauf et al., 2002). These cells were cultured in Dulbecco's Modified Eagle Medium (DMEM) supplemented with 10% Fetal Bovine Serum (FBS), 1% penicillin, 1% streptomycin, 1% L-glutamine (PSLG), 100 μg/ml geneticin and 0.4 μg/ml puromycin. To induce Cx43YFP expression, cells were incubated with 1 μg/mL doxycycline. Wild type HeLa cells were cultured in DMEM supplemented with 10% FBS and 1% PSLG. Cell media was changed every 48-72 hours. Cells were incubated at 37° C. with 5% CO₂. All studies were conducted at least five days after plating at 80% confluency.

Optical Microscopy.

Fluorescence and brightfield images have been optimized for contrast and brightness. A Zeiss AxioObserver microscope with 10× and 20×, numerical aperture (NA) 0.25 and 0.8 objectives was used for widefield imaging. A Zeiss AxioObserver Spinning Disk Confocal microscope with a 100× oil immersion (NA 1.4) and a 63× oil immersion (NA 1.4) objective was used for both fluorescence and brightfield imaging. Three filters were used: an emission filter centered at 525 nm with a 50 nm width, an emission filter centered at 629 nm with a 62 nm width, and a triple pass dichroic mirror designed to reflect laser illumination at 405 nm, 488 nm, and 561 nm excitation wavelengths. For spinning disk confocal and brightfield imaging, cells were cultured on 35 mm collagen-coated glass bottom dishes (MatTek).

Flow Cytometry.

A BD Accuri C6 Flow Cytometer was used for all flow cytometry studies and all flow cytometry data was analyzed using FlowJo software. Flow cytometry data was collected at a speed of 35 events per second. Gates were drawn to include at least 30% of the detected events. In each experiment, once the appropriate gate was determined it was applied to all trials and all experimental conditions without modification.

Giant Plasma Membrane Vesicle (GPMV) Formation.

GPMVs were formed by rinsing donor connexin 43-YFP HeLa cells twice with GPMV buffer (2 mM CaCl₂, 10 mM HEPES, 150 mM NaCl) and once with active buffer (2 mM CaCl₂, 10 mM HEPES, 150 mM NaCl, 25 mM PFA, 2 mM DTT, 125 mM glycine). Then, the cells were incubated for 6 hours in active buffer, and the active buffer containing the vesicles was collected from the cells. To concentrate, the GPMVs were centrifuged at 17,000×g for 30 minutes at 4° C. Finally, the GPMV pellet was resuspended in fresh GPMV buffer. To determine average GPMV diameter, the diameters of 154 vesicles were measured from brightfield images. To determine the average percentage of GPMVs containing connexin 43-YFP embedded in the membrane, confocal spinning disk and brightfield images of 3 independent batches of vesicles were analyzed. At least 37 GPMVs were analyzed per batch.

YFP Purification.

The pET28a-HisYFP-Sp100 plasmid was from the lab of Frauke Melchior (Addgene plasmid #53141) (Flotho et al., 2012). Following the provider's protocol, the YFP-Sp100 was expressed in BL21(DE3)pLysS cells for 1 hour at 18° C. and then 5 hours at 30° C. Bacterial extracts were made by lysing the cells in 50 mM Na₃PO₄, pH 8.0, 300 mM NaCl, 20 mM imidazole, 1 mM β-ME, and 1 μg/mL each of leupeptin, pepstatin, and PMSF. HisYFP-Sp100 was purified by incubating with Ni-NTA agarose beads. After washing with 50 mM Na₃PO₄, pH 8.0, 300 mM NaCl, 20 mM imidazole, 1 mM β-ME, and 1 μg/mL each of leupeptin, pepstatin, and PMSF, proteins were eluted in 250 mM imidazole. Eluted proteins were concentrated and dialyzed in 50 mM Na₃PO₄, pH 8.0, 300 mM NaCl, 1 mM β-ME at 4° C. overnight followed by a second 2 hour dialysis at 4° C. The final protein concentration was calculated from the absorbance spectrum measured on a Nanodrop 2000 spectrophotometer (Thermo Scientific).

Quantification of Connexon Density on GPMV Surfaces.

Purified SP100-YFP was serially diluted to generate a calibration curve of protein concentration. The YFP fluorescence of the calibration curve and a sample of 8.9×10⁶ vesicles were measured in a BioTek Cytation 3 fluorimeter to calculate the average molar concentration of YFP molecules in the vesicle sample. The molar concentration was converted to number of YFP molecules and divided by the number of vesicles per sample to achieve an average number of connexin 43-YFP molecules per vesicle. Finally, the surface area per vesicle was calculated from the average diameter described above to determine a density of connexons per vesicle. The percentage of the vesicle surface covered by connexons was estimated using the approximate membrane area per connexon channel in cellular membranes (Unwin and Zampighi, 1980), 72.25 nm².

Calcein Red-Orange Loading.

A stock solution of calcein red-orange dye in DMSO was prepared at a concentration of 1.7 mg/mL and diluted to a final concentration of 17 ng/μL in GPMV buffer. To form calcein red-orange GPMVs, donor cells were incubated in the dye solution for 30 minutes immediately before GPMV formation.

Connexon Function Study.

For the calcein red-orange release study, GPMVs containing calcein red-orange were formed from donor cells as described above. Connexon channels were opened by removing calcium from the solution with a final concentration of 5 mM EGTA and EDTA. GPMVs were imaged within 2 hours of EGTA and EDTA addition, and examined for luminal fluorescence above the background level. At least 54 GPMVs were analyzed for each trial.

For the Atto 594 release study, a 10 μM stock solution of Atto 594-NHS Ester (with NHS ester hydrolyzed) was prepared in GPMV buffer. Atto 594 was added to preformed GPMVs in GPMV buffer at a final concentration of 20 nM. To remove calcium from solution, EGTA and EDTA were added to the vesicles as described above. GPMVs were imaged within 1 hour of EGTA and EDTA addition, and examined for luminal fluorescence above the background level. At least 51 GPMVs were analyzed for each trial.

For the photobleaching study, Atto 594 dye within GPMVs was bleached using a 561 nm laser on the spinning disk confocal microscope described above. Then, laser illumination was stopped and images were taken every 15 seconds for 75 seconds.

Dye Delivery Study.

Recipient HeLa cells were plated in a 12 well plate at a density of 25,000 cells per well and a total media volume of 2 mL per well. Seven hours before addition of the GPMVs, the recipient cells received fresh media. The media for the blocked condition was supplemented with 300 μM carbenoxolone, prepared from a 100 mM stock in water. Just before addition of the GPMVs, the recipient cells were rinsed once with 2 mL PBS and then incubated in 500 μL fresh PBS. Recipient cells for the blocked condition were incubated in PBS supplemented with 300 μM carbenoxolone. Calcein red-orange-loaded vesicles were formed using calcium-free GPMV buffer. The GPMVs were resuspended in fresh calcium-free GPMV buffer and counted with a hemocytometer. GPMVs were added to recipient cells at a ratio of approximately 1 GPMV per 2 recipient cells and incubated in the dark at 37° C. and 5% CO₂ for 2 hours. This ratio was estimated based on hemocytometer counts of the number of vesicles and of the number of recipient cells in a 12 well dish. After incubation, the recipient cells were rinsed with 2 mL PBS to remove the vesicles and then imaged or prepared for flow cytometry. For flow cytometry, the recipient cells were trypsinized with 500 μL trypsin for 5 minutes at 37° C., 5% CO₂ and then quenched with 1500 μL media and centrifuged for 5 minutes at 300×g. The cell pellet was resuspended in 100 μL PBS before flow cytometry. At least 7,500 cells were analyzed for each trial.

Doxorubicin Loading.

A 10 mM stock solution of doxorubicin was prepared in DMSO. To form doxorubicin-loaded vesicles, donor cells were incubated in a 1 mM doxorubicin solution in calcium-free active buffer for 30 minutes immediately before GPMV formation. After 30 minutes, the doxorubicin was diluted to a final concentration of 200 μM in active buffer without calcium and remained throughout the duration of blebbing. After formation, doxorubicin-loaded GPMVs were collected as described above and then washed with 1 mL fresh GPMV buffer without calcium. GPMVs were then resuspended in fresh GPMV buffer without calcium.

Doxorubicin Encapsulation Measurement.

To estimate the amount of doxorubicin encapsulated within the GPMVs, free doxorubicin was serially diluted to generate a calibration concentration curve (n=3). The doxorubicin fluorescence of the calibration curve, 3 samples of 300,000 doxorubicin-loaded vesicles, and 3 samples of 300,000 empty vesicles was measured in a BioTek Cytation 3 fluorimeter to determine the average molar doxorubicin concentration of the GPMV samples. This molar concentration was converted to moles of doxorubicin and divided by the approximate total volume of all GPMVs (based on average concentration and diameter of GPMVs) present in the sample to estimate the concentration of doxorubicin inside the GPMVs.

Doxorubicin Retention Study.

Doxorubicin-loaded GPMVs were imaged with the spinning disk confocal microscope immediately following formation and again after 2 hours. The relative change in the Doxorubicin content of the GPMVs was estimated by measuring the average fluorescence intensity within the vesicles. Thirty GPMVs were analyzed for each condition.

Doxorubicin Release Study.

The initial doxorubicin content of the GPMVs was measured using flow cytometry. EGTA and EDTA were added to the GPMV solution as described above in the connexon function studies in order to open the connexon channels. Using flow cytometry, the final doxorubicin content of the GPMVs was then measured 5 minutes after EGTA and EDTA addition. While some GPMVs ruptured upon addition of EGTA and EDTA, at least 850 GPMVs were analyzed for each trial.

Doxorubicin Cytotoxicity Study.

Recipient HeLa cells were plated in a 12 well plate at a density of 25,000 cells per well and a total media volume of 2 mL media per well. Seven hours before addition of the GPMVs, recipient cells received fresh media. Just before addition of the GPMVs, the recipient cells were rinsed once with 2 mL PBS and then incubated in 500 μL fresh PBS. Concentrated GPMVs were resuspended in fresh calcium-free GPMV buffer and counted using a hemocytometer. GPMVs were added to recipient cells at a ratio of approximately 1 GPMV per 2 recipient cells and incubated in the dark at 37° C. with 5% CO₂ for 2 hours. After incubation, the recipient cells were rinsed with 2 mL media and then incubated in 2 mL fresh media at 37° C. with 5% CO₂. After 24 hours, cell viability was analyzed using a trypan blue or 7-AAD permeability assay.

For the trypan blue assay, the cells were trypsinized with 500 μL trypsin for 5 minutes at 37° C., 5% CO₂. Trypsinized cells were then quenched with 1.5 mL media and centrifuged for 5 minutes at 300×g. The cell pellet was resuspended in 200 μL media, and trypan blue was added to the cells at a volume ratio of 1:1. At least 90 cells were counted for each trial using a hemocytometer. Cells including the trypan blue stain were considered non-viable and cells excluding the trypan blue were considered viable.

For the 7-AAD assay, the cells were trypsinized with 500 μL trypsin for 5 minutes at 37° C., 5% CO₂. Trypsinized cells were then quenched with 1.5 mL media and centrifuged for 5 minutes at 300×g. The cell pellet was resuspended in 100 μL PBS. Five μL of 7-AAD was added to 45 μL of the resuspended cells, and analyzed using flow cytometry within three minutes of 7-AAD addition. At least 5,000 cells were analyzed for each trial. Cells including the 7-AAD stain were considered non-viable and cells excluding the 7-AAD were considered viable. To determine the percentage of non-viable cells in each sample, a threshold was drawn on the flow cytometry fluorescence histograms at the minimum point between the population of cells excluding the dye and the population of cells including the dye. The percentage of cells with fluorescence above these thresholds were considered non-viable.

The relationship between the concentration of doxorubicin added via solution and cell viability was determined by incubating recipient HeLa cells in the specified doxorubicin concentration diluted in media from a 10 mM stock in DMSO for 24 hours. Cell viability was measured using both viability assays as described above.

### Example 4—Rebuilding the Gap Junction Network

A substantial benefit of the methods provided herein is the opportunity to rebuild the gap junction network within tumors. Overwhelming evidence supports the role of gap junctions as tumor suppressors (Naus et al., 2010), including (i) down-regulation of connexins in breast cancer cells (Shao et al., 2005), (ii) inhibition of gap junctions by many carcinogens (Mesnil, 2002), (iii) reduced proliferation of cancer cells when gap junctions are expressed (Eghbali et al., 1991), and (iv) increased tumor onset and metastasis in animals lacking gap junctions (Avanzo et al., 2004). Unfortunately, cancer cells down-regulate junction proteins, enabling rapid cell division (Yamasaki et al., 2004), epithelial to mesenchymal transition, and metastasis (Li et al., 2004). Thus, experiments were performed to simultaneously access the cellular cytoplasm and rebuild gap junction networks within tumors with Connectosomes.

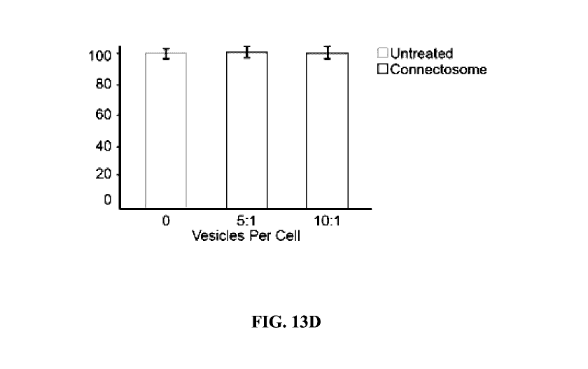

Connectosomes were extracted from the plasma membrane of HeLa donor cells overexpressing CX43-YFP proteins (FIG. 12A). The unprocessed Connectosomes ranged in diameter from 3.5 to 13.4 μm, with an average diameter of 7.5 μm, as assessed by confocal imaging (FIG. 12B). Over 63% of the Connectosomes contained CX43-YFP at levels detectable by fluorescence imaging. Using the same blebbing process, GPMVs were obtained from A549 lung cancer and HeLa cells. The resultant GPMVs did not significantly differ in morphology or size as a function of cell type. A549 cells are known to naturally express very low levels of connexin, while wild type HeLa cells express lower connexin levels as compared to the overexpressing CX43-HeLa cell line. Therefore, A549 and HeLa cells were chosen for the production of GPMVs with reduced levels of connexin proteins to be used as a negative control.

Connectosomes were used either as obtained from cells (unprocessed) or after extrusion through a 1 μm polycarbonate membrane, to obtain a more uniform vesicle population size distribution (FIG. 12C). To more easily visualize the extruded GPMVs, highly fluorescent Connectosomes were prepared by staining the donor cells with Texas Red DHPE immediately before blebbing (FIG. 12C). The average size of the extruded Connectosomes was submicron.

In order to verify the biological activity of the connexin embedded in the GPMVs, the innate calcium-induced gating of the connexon channels was used. Specifically, calcium induces closure of connexon channels, while the absence of calcium causes a conformational change that opens the channel. Donor cells were treated with CRO AM dye. Since the dye can freely diffuse through cell membrane, the cells were easily loaded with the dye and when they were subsequently blebbed, GPMVs loaded with the CRO AM were obtained. However, when in the cell cytoplasm the acetomethoxy group of CRO AM dye is hydrolyzed, making the dye membrane impermeable. The CRO dye inside the GPMV can therefore diffuse out only through opened membrane channels. As expected, the connexons in the dye-loaded Connectosomes opened upon calcium depletion, releasing the CRO dye encapsulated within the GPMVs (FIG. 12D, F). These results confirm the presence of functional connexons in the GPMV membrane. In contrast, the GPMVs extracted from A549 cells did not release encapsulated dye upon calcium treatment, confirming that blebs from A549 cells have much lower levels of functional connexin channels in comparison to Connectosomes (FIG. 12E, F).

Next, it was evaluated whether Connectosomes could interact with MDA-MB-231 breast cancer cell cultures and reduce cell migration via reinforcement of junctional networks. Imaging the recipient MDA-MB-231 cells after 4 hours of incubation with the extruded Connectosomes, showed the accumulation of GPMVs at the cell membrane and at cell junctions (FIG. 12G-H). The impact of connexin43 expression on the interaction between Connectosomes and recipient cells was also measured by using flow cytometry to quantify the fluorescence of MDA-MB-231 cells exposed to either TR-labeled Connectosomes and TR-labelled HeLa cell derived GPMVs. As shown in FIGS. 12I and J, treating MDA-MB-231 with Connectosomes resulted in a significantly higher average florescence intensity per cell compared to the untreated control cells and cells treated with HeLa cell derived GPMVs. These results demonstrate that expression of connexin 43 proteins on the membrane surface enhances adhesion of the GPMVs to cells, suggesting that GPMVs are forming gap junctions with MDA-MB-231 cells.

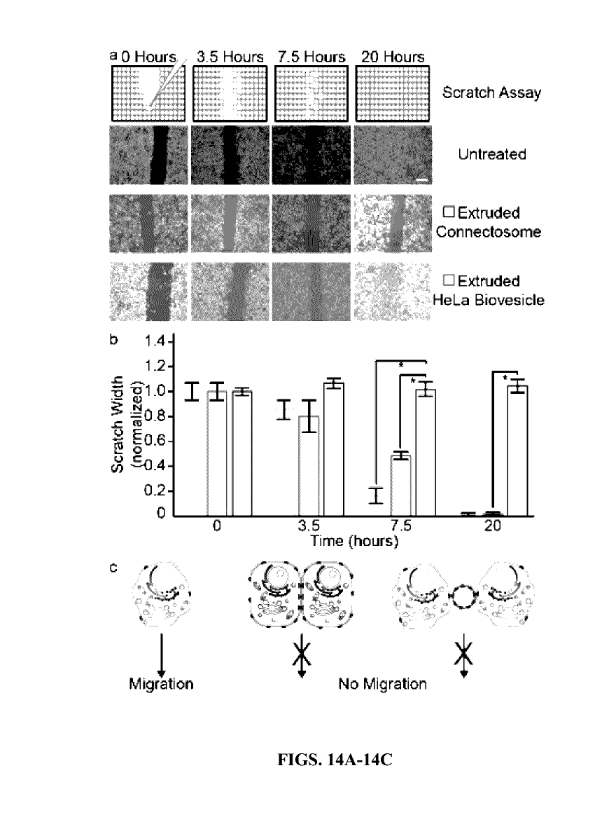

Having established the connexin-dependent association between Connectosomes and cells, an MTT proliferation assay (FIG. 13D) was performed to determine whether Connectosomes, either unprocessed or extruded, were toxic to cells. These results indicated that Connectosomes dosed at 5 and 10 Connectosomes per cell did not negatively impact the proliferation of MDA-MB-231 cells as compared to control untreated cells, supporting the absence of adverse effects triggered by the GPMVs. The migration studies presented below were performed at Connectosomes to cell ratios that did not exceed these levels.

The ability of the Connectosomes to influence migration of metastatic MDA-MB-231 cells was assessed with both a migration assay and a scratch healing assay. Cells were starved for 24 hours and then seeded in serum free medium on a permeable Transwell™ membrane, pre-coated with basement membrane extract (BME), mainly composed of laminin and collagen IV. Cells were treated with extruded Connectosomes at different concentrations and medium with serum was placed in the lower compartment as a chemoattractant to stimulate cell migration in one direction. After 24 hours, cells that invaded the BME and migrated through the membrane were stained with crystal violet and counted (FIG. 13A). As reported in FIG. 13, exposure of cells to Connectosomes led to a concentration dependent reduction in the migration of cancer cells. For low ratios, 0.025:1 and 0.05:1 (GPMVs per cells), the impact on migration was minimal, but for ratios above 0.1:1, the decrease became statistically significant (p<0.05), with a 50% reduction in cell migration for the ratio 0.2:1.

The impact of Connectosomes on cell migration was compared to the impact of GPMVs extracted from HeLa cells, which possess reduced levels of connexin as compared to Connectosomes. A significantly less pronounced effect (P<0.01) on cell migration was observed when MDA-MB-231 cells were treated with regular HeLa extruded GPMVs at 0.4:1 and 5:1 GPMVs per recipient cell ratios (FIG. 13). Since the only difference between Connectosomes and HeLa GPMVs is the overexpression of CX43 in Connectosomes, these results demonstrate that the reduction in cell migration associated with exposure to Connectosomes is dependent on CX43 expression.

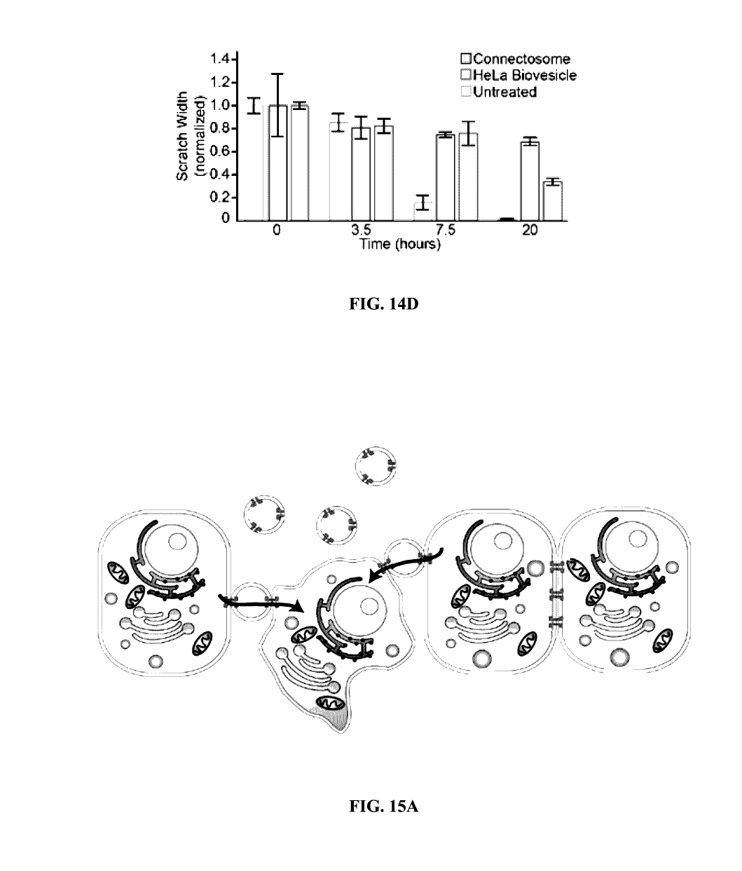

The decrease in migration upon treatment with Connectosomes was further verified with a scratch healing assay, in which the impact of Connectosomes and control GPMVs on the closure of a scratch was measured in a confluent cell monolayer. While the transwell migration assay measures the ability of cells to invade through a BME extract and migrate toward a chemoattractant, the scratch assay does not require BME or chemoattractant and therefore measures the ability of a monolayer to close a perturbation of integrity by directed migration, proliferation, and cell spreading, to restore the cell-cell interactions. To perform the assay a scratch was created through a confluent monolayer of MDA-MB-231 cells. The width of the scratch, normalized to the original width, was recorded at fixed time points and used as a measure cell migration rate. Untreated control cells were able to migrate and close the scratch within 20 hours with no visible scratch remaining in the cell monolayer (FIG. 14). Interestingly, migration of the MDA-MB-231 cells treated with both unprocessed and extruded Connectosomes (at a ratio of 10:1 GPMVs per cell) was reduced, with a significant decrease in migration already appreciable at 7.5 hours (P<0.05) and the scratch still visible at 20 hours post treatment (FIG. 14).

Consistent with the migration assay, GPMVs from regular HeLa cells elicited almost no effect compared to the Connectosomes due to the reduced expression of connexin (FIG. 14). Specifically, exposure to HeLa cells delayed scratch closer only slightly with a 48% open scratch at around 7.5 hours as compare to a 39% of control, while exposure to Connectosomes results in no significant closure of the scratch even after 20 hours of exposure. It is interesting to note that cells treated with Connectosomes appear more round and less adhered to the well substrate, as compared to control cells. A negative feedback between cell-matrix adhesion and cell-cell interaction is well established in the field, and reduced cell-matrix interactions are known to be associated with reduced traction and cell motility (Brute and Thery, Current Opinions in Cell Biology, 24(5):628-636, 2012). Therefore, reinforcement of cell-cell interactions by Connectosomes could have potentially weakened cell-matrix adhesion, leading to the observed reduced migration.

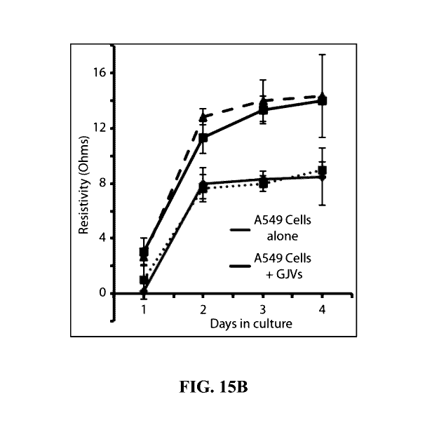

Finally, lung cancer cells A549 that are known to express very little connexin and therefore a non-tight monolayer. The A549 cells were exposed to increasing concentrations of GPMVs and trans-epithelial electric resistance (TEER) which correlates with the tightness of the monolayer was found to be increased (FIG. 15).

### Example 5—Targeted GPMVs

Targeting GPMVs specifically to tumor cells, such as breast tumor cells, is a critical step toward improved drug delivery. Triple negative breast tumor cells are known to over-express the epidermal growth factor receptor (EGFR). Therefore, nanobodies (single-domain antibody fragments) against EGFR (Roovers et al., 2007) were expressed on the surfaces of the donor cells used to make GPMVs. Notably, this strategy could be used to express a broad range of protein ligands enabling targeting to many types of cancerous cells. Expression of these ligands can reach the concentration required for efficient targeting (Kirpotin et al., 1997). The targeted GPMVs were loaded with doxorubicin using the procedures in Example 2. To determine the selectivity of targeted doxorubicin-loaded GPMVs the percent cell viability was mapped as a function of dose for cells expressing EGFR (MDA-MB-231 and MDA-MB-468) and for cells that do not express EGFR (MCF-7 cell line (Mamot et al., 2003)), as well as MDA-MB-231 cells in which EGFR had been knocked down. The expression level of ligands was varied to determine the optimum level for specific drug delivery. Control experiments using targeted GPMVs without encapsulated drugs ensured that cell killing was not the result of ligand-receptor interactions. Experiments in the presence of the gap-junction blocker, Carbenoxolone (Ye et al., 2009), ensured that cell killing was not the result of endocytic uptake of GPMVs. Collectively, these studies optimized GPMVs for specific targeting of cells that overexpress EGFR.

Design and Expression of Chimeric Transmembrane Proteins for Cellular Targeting:

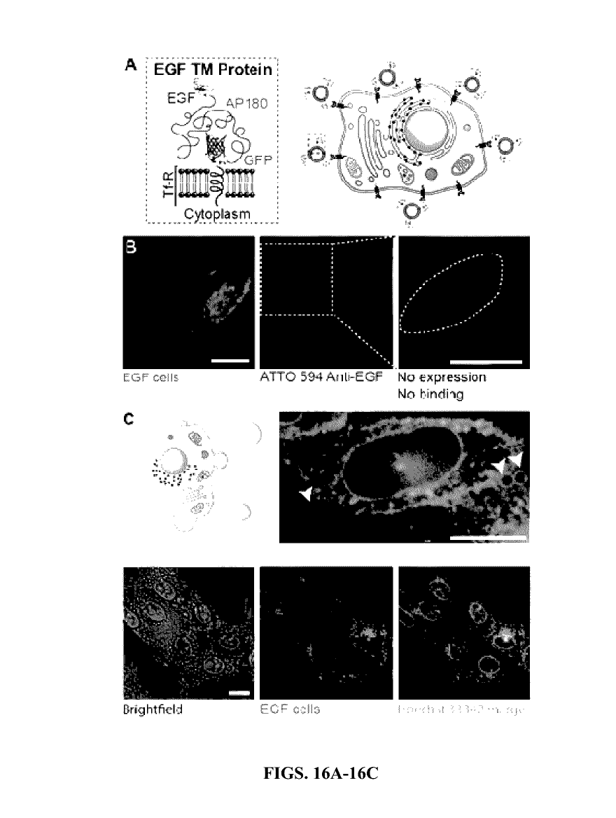

The model receptor targeted was the Epidermal Growth Factor Receptor (EGFR). Multiple human cancers including breast, non-small cell lung cancer, ovarian and colorectal cancer, express EGFR at elevated levels, making EGFR a popular target for molecular delivery to tumors. To precisely target cells on the basis of EGFR expression level, a chimeric targeting protein was designed that consisted of the intracellular and transmembrane domains of the transferrin receptor. The ectodomain of the chimeric protein consisted of an eGFP domain followed by the first 289 amino acids of the intrinsically disordered C-terminal domain of the intracellular protein AP180, and finally a targeting moiety, either EGF or a single domain antibody against EGFR (FIG. 16A). eGFP enables direct visualization and tracking of the targeting protein using a fluorescence microscope, while the intrinsically disordered domain, much like the polyethylene glycol (PEG) polymers on synthetic liposomes, provides a flexible linker and spacer between the targeting ligands and the surface of the lipid bilayer, enabling the ligands to interact with the targeted cell surface receptors. The expected hydrodynamic radius of the intrinsically disordered linker is approximately 3.8 nm, similar to a PEG 5000-10000 chain.

The cell-surface expression of the EGF targeting protein was tested using a live cell, fluorescence-based antibody-binding assay. CHO cells transiently expressing the EGF targeting protein showed a robust eGFP signal at the cellular plasma membrane (FIG. 16B left). Binding of ATTO 594 labeled-EGF antibodies to the cell surface demonstrated that EGF was expressed on the extracellular leaflet of the phospholipid bilayer (FIG. 16B middle). In addition, cells from the same culture dish with little or no expression of the eGFP-tagged targeting protein did not recruit antibodies against EGF, further confirming the specific binding between the antibodies and the chimeric targeting protein (FIG. 16B right). Notably, the GFP-tagged EGF targeting proteins are translated and produced in the endoplasmic reticulum before being transported to the Golgi apparatus for post-translational modifications, and eventually trafficked onto the plasma membrane of the cells. Therefore, the fluorescence signal of the GFP-tagged targeting protein is expected to exist throughout the cell interior, as observed in the left panel of FIG. 16B. In contrast, the antibody binds from the outside of the cell and is therefore expected to be present primarily on the outer cell surface, though uptake of the antibody during receptor recycling produces some internal antibody signal, as shown in FIG. 16B.

Following the expression of functional targeting protein, GPMVs were extracted from these donor cells (FIG. 16C). After the extraction process the donor cells remained attached to the culture dish and fluorescent images suggested that they had similar morphological appearances to normal donor cells. Hoechst 33342 staining showed that the nuclei of the donor cells remained intact (FIG. 16D) and the GPMVs were free of nuclear contamination.

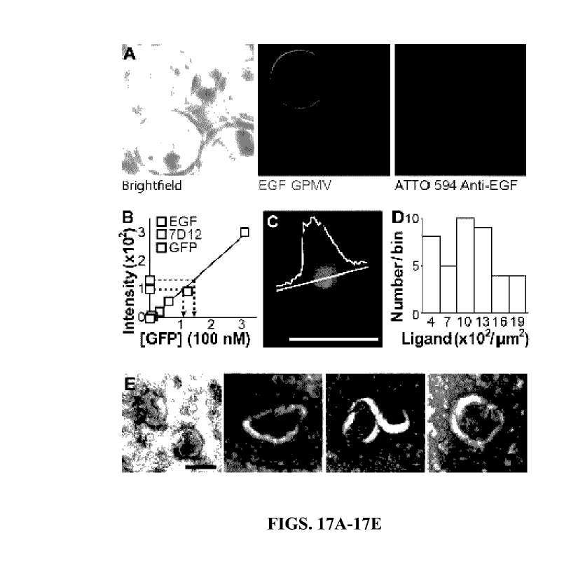

To test whether targeting proteins on GPMV surfaces are able to engage in molecular binding, ATTO 594 labeled antibodies against EGF were incubated with GPMVs. Anti-EGF bound to the surfaces of GPMVs that displayed the EGF targeting proteins (FIG. 17A). In contrast, the labeled antibodies did not bind to GPMVs that lacked a significant eGFP signal, indicating lack of significant expression of the EGF targeting protein (FIG. 17A, left and right). Fluorescence intensity analysis of the eGFP and ATTO 594 signals demonstrated a correlation between the display of the targeting protein and the extent of antibody binding. Taken together, these data demonstrate that GPMVs extracted from donor cells expressing the EGF targeting protein displayed the targeting protein on their surfaces such that the ligand domain was accessible to the external solution.

Quantifying the Density of Targeting Proteins on PMV Surfaces:

Several studies have shown that increasing the density of ligands on the surfaces of targeted particles can significantly increase nanoparticle binding to target cells, increasing the cell-particle binding affinity by as much as 10-fold. Therefore, having a sufficient density of ligands on the surfaces of targeted particles is critical to achieving high affinity binding. To estimate the density of targeting proteins displayed on the surfaces of GPMVs, two distinct fluorescence-based approaches were developed. The first is based on measuring the calibrated total fluorescence of the GPMV sample normalized by an estimate of its total membrane content, while the second is based on calibrated fluorescence intensity measurements of individual GPMVs. Conventional methods were used to produce a stable cell line expressing the EGF targeting protein. Notably, more than 80% of the stably transfected cells expressed significant levels of the targeting proteins, as demonstrated by elevated fluorescence intensity in the GFP channel during flow cytometry-based characterization. Expression of the EGF targeting protein was confirmed by immunoblotting GPMVs with an antibody against EGF.

First, based on the total fluorescence of GPMVs in solution and an average GPMV diameter of 11 μm, it was determined that there were on average 400 copies of the EGF targeting proteins per square micrometer of the vesicle surface (FIG. 17B). It was estimated that each targeting protein occupies an area of 50 nm² on the membrane surface, based on a worm-like chain model of the intrinsically disordered domain. Combining this estimate of the area per protein with the measured density of targeting proteins on the membrane surface, the EGF targeting proteins cover approximately 2% of the total membrane surface. The autofluorescence of GPMVs derived from CHO cells without GFP expression was also measured and found to be small in comparison to the GFP signal.

As a second estimate of ligand density, a quantitative fluorescence microscopy assay was employed on individual GPMVs. In comparison to the bulk method described above, a higher density of targeting proteins was expected from this assay since GPMVs that lack significant eGFP fluorescence intensity cannot be clearly visualized on the basis of fluorescence and are thus under-represented in the analysis. To calculate the number of targeting proteins displayed per diffraction-limited unit of membrane area, the mean fluorescence intensity of the GPMV surface was divided (FIG. 17C) by the integrated brightness of a single eGFP molecule. Forty total GPMVs from 3 independent sample preparations yielded an average of 1200 (400-2200) copies of the EGF targeting protein per square micrometer (FIG. 17D). Notably, both measures of targeting protein density fall within or above the range cited above from the work of Nielsen et al. and are therefore expected to provide robust targeting of plasma membrane vesicles. The substantial variation in the targeting protein density among GPMVs likely arises from variation in targeting protein expression among the donor cells, suggesting that sorting or gene editing of the donor cells would provide a more uniform targeting protein density.

EGFR Targeting is Sensitive to Cellular Receptor Expression:

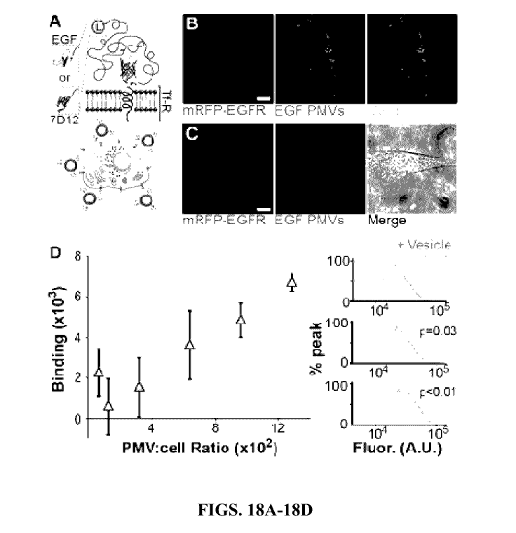

To evaluate cell targeting, GPMVs were extruded through one-micrometer polycarbonate filters to produce plasma membrane vesicles (PMVs). Vesicles of this size are convenient for targeting studies because they are small enough to avoid gravitational settling yet large enough to track easily using fluorescence microscopy. However, PMVs can be further extruded through 100 nm filters to produce a homogenous population of vesicles of the appropriate size for in vivo studies. Transmission electron micrograph images conveyed that PMVs have similar morphology to other liposomal particles (FIG. 17E). To investigate the ability of PMVs to target specific cells (FIG. 18A), PMVs expressing the EGF targeting protein were incubated with HeLa cells transiently expressing mRFP-tagged EGFR. At the end of the incubation following repeated washing of the cells, there was extensive colocalization of PMVs (eGFP signal) with cells overexpressing mRFP-tagged EGFR (FIG. 18B). In contrast, PMVs bound much less strongly to cells in the same culture dish that lacked a significant mRFP-EGFP signal (FIG. 18C). Notably, HeLa cells express EGFR endogenously, such that some binding of EGF-PMVs to all cells was expected.

The amount of PMV binding to cells with a high endogenous level of EGFR (MDA-MB-468 cells) was quantified as a function of increasing PMV concentration. Specifically, the shift in fluorescence intensity in a spectral region corresponding to eGFP was quantified using flow cytometry (FIG. 18D right). Cells were incubated with EGF-PMVs at a range of concentrations for 4 hours at 37° C. The cells were carefully washed 3 times with PBS to remove unbound PMVs and then trypsinized for flow cytometric analysis. As the concentration of PMVs increased, binding to MDA-MB-468 cells also increased (FIG. 17D left), indicating a positive correlation between PMV dosage and binding.

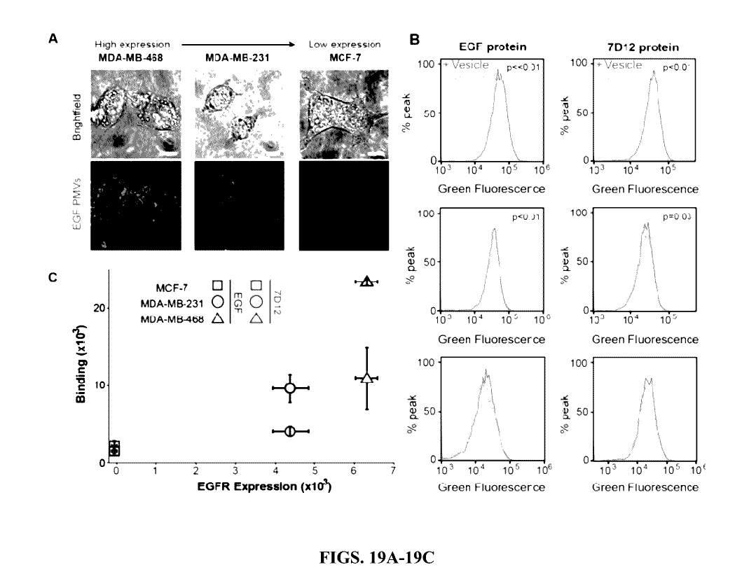

To further confirm the specificity of EGF-PMVs for EGFR expressing cells, three breast cancer lines (MCF-7, MDA-MB-231, and MDA-MB-468) were used which have increasing endogenous levels of EGFR expression. This trend of increasing EGFR expression was confirmed using flow cytometry studies on cells exposed to a fluorescent-labeled antibody against EGFR, obtaining a trend consistent with literature values. Following this confirmation, the three cell types were individually incubated with EGF-PMVs. PMV bound cells were first visualized using fluorescence confocal microscopy. As expected, the PMVs bound most abundantly to the MDA-MB-468 cells, which had the highest EGFR expression level, while they bound least to the MCF-7 cells, which had the lowest EGFR expression level (FIG. 19A). To quantify the amount of binding, cells that had been incubated with PMVs were washed and analyzed using flow cytometry. The results from these experiments confirmed an increasing level of EGF-PMV binding to the cells as the expression level of EGFR increased, demonstrating that EGF-PMVs are sensitive to EGFR expression level (FIGS. 19B and 19C).

As an alternative to the EGF ligand, PMVs were also developed that used the 7D12 nanobody against EGFR as the targeting ligand. This choice of targeting ligand is more appropriate for therapeutic applications, since it lacks the potential mitogenicity of a growth factor. Specifically, nanobodies, single-domain antibodies derived from camelids, have nanomolar binding affinities and are much smaller in size, (˜15 kDa), in comparison to conventional antibodies, averaging around 150 kDa. They have emerged as a useful tool for cellular targeting, as studies have shown that gold nanoparticles chemically conjugated to nanobody against human epidermal growth factor receptor 2 (HER2) bound selectively to HER2 overexpressing cells. The 7D12 nanobody binds EGFR with high affinity and blocks downstream EGFR signaling. As such, the 7D12 nanobody provides an alternative to the EGF ligand for targeting EGFR positive cells (FIG. 18A). 7D12-PMVs were prepared from a CHO cell line stably expressing the 7D12 targeting protein, using the same procedures used to prepare EGF-PMVs. The density of the 7D12 targeting protein on PMVs was somewhat lower in comparison to expression of the EGF targeting protein, with an average of over 300 copies per square micrometer (based on the ensemble assay), yielding a total surface coverage of approximately 1.6% (FIG. 17B).

When incubated with each of the three breast cancer cell lines (MCF-7, MDA-MB-231, and MDA-MB-468), 7D12-PMVs behaved similarly to EGF-PMVs, showing a trend of increasing binding with increasing EGFR expression level (FIGS. 19B and 19C), though the absolute fluorescence values in the flow cytometry studies were somewhat lower. The reduced signal from 7D12-PMVs likely resulted from two factors. First, 7D12, has a reported dissociation constant for EGFR binding of 200 nM, which is substantially higher than the dissociation constant for EGF binding to EGFR, 5 nM, indicating weaker binding. Further, the density of the 7D12 targeting protein was approximately 30% less than the density of EGF targeting proteins, as noted above (FIG. 17B). Nonetheless, 7D12-PMVs demonstrated clear sensitivity to EGFR expression level.

Targeting Cells that Express GFP-Tagged Receptors:

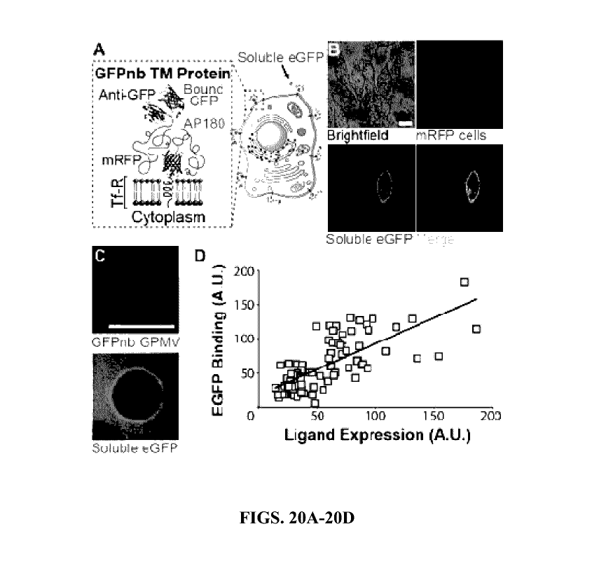

These studies demonstrated selective binding of PMVs on the basis of EGFR expression level using two different targeting ligands. To evaluate whether this strategy can be extended to an arbitrary receptor, a targeting protein was developed that selectively binds to any GFP-tagged receptor. The ligand domain of this targeting protein is a single domain antibody that specifically recognizes GFP (FIG. 20A). For this targeting protein the fluorophore domain consisted of mRFP, rather than eGFP, so that the targeting protein and its ligand (GFP) would have distinct fluorescent signatures. Creating PMVs that target GFP-tagged receptors provides an opportunity to evaluate the absolute specificity of PMVs for target cells, since cells lack endogenous GFP expression. Further, the ability to target PMVs to cells that express GFP-tagged receptors could be useful for molecular delivery to engineered cell lines in complex contexts such as engineered tissues and cell implantation studies, where engineered cells are surrounded by other cell types.

Following the expression of the GFPnb targeting protein by donor cells, the external accessibility and functionality of the targeting ligand was tested. The GFPnb targeting protein was transiently expressed in CHO cells and then incubated these with soluble eGFP (FIG. 20B). The soluble eGFP bound significantly only to cells expressing the GFPnb targeting protein and was not recruited by cells in the same dish that lacked significant expression of the targeting protein. Further, GPMVs derived from CHO cells stably expressing the GFPnb targeting protein were also capable of recruiting soluble eGFP from solution (FIG. 20C), confirming that the GFP nanobody on the surfaces of GPMVs was accessible to the external environment and able to bind to eGFP. Fluorescence intensity analysis of the GPMVs and the soluble eGFP revealed a correlation between the expression of the GFPnb targeting proteins and the amount of soluble eGFP binding (FIG. 20D).

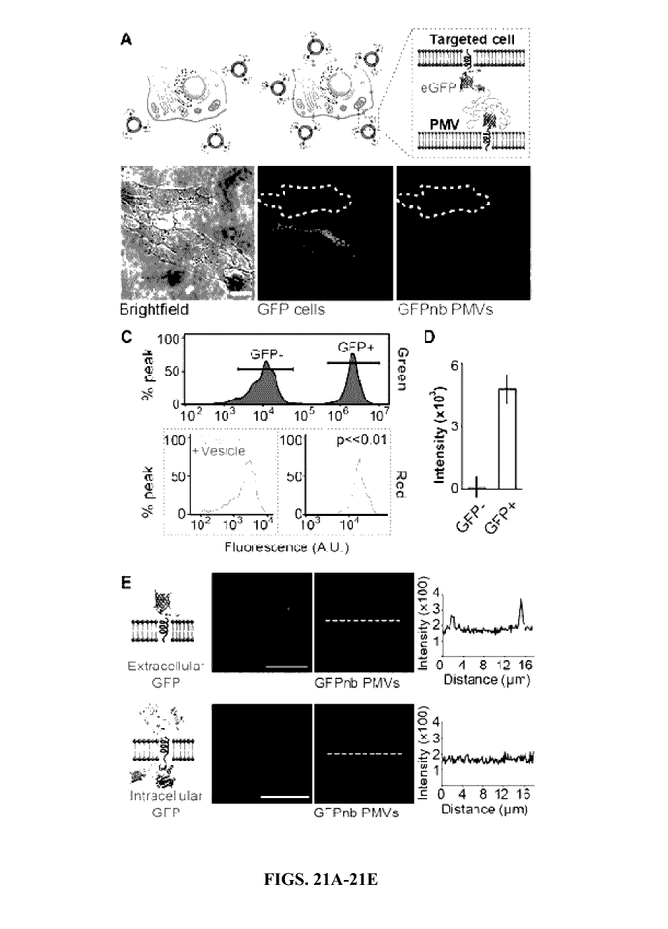

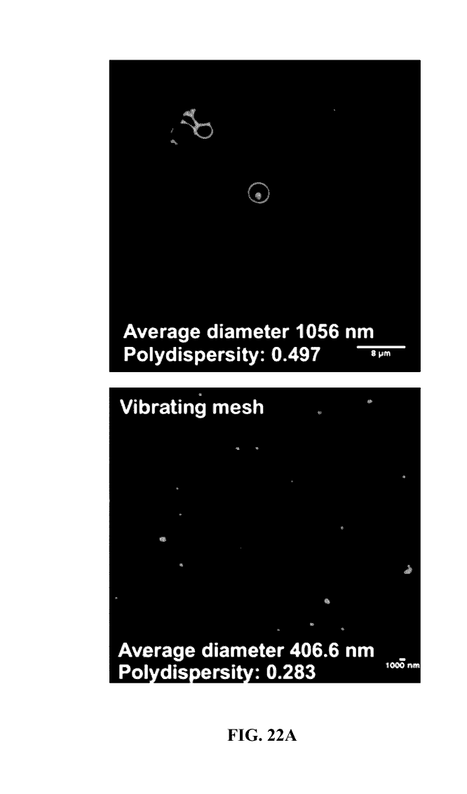

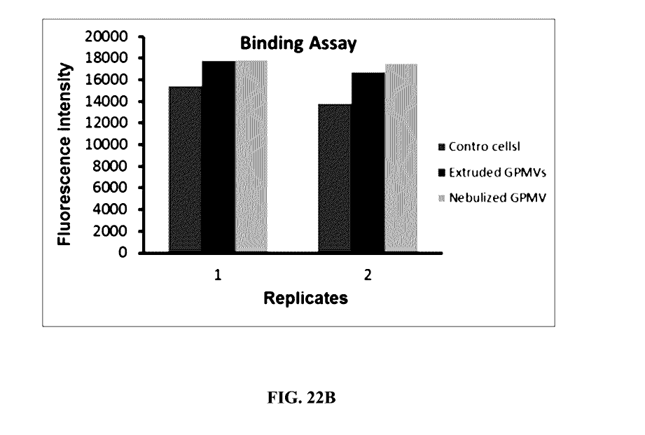

Next, the ability of GFPnb-PMVs to target eGFP-expressing cells was evaluated. In particular, a competitive binding assay was conducted where CHO cells stably expressing eGFP on the cell surface (GFP positive cells) and CHO wild type cells (GFP negative cells) were co-cultured in a single culture dish (FIG. 21A). At the end of incubation, extensive colocalization between PMVs and the cell membrane of GFP positive cells was observed. In contrast GFP negative cells bound significantly fewer PMVs (FIG. 21B). This experiment was repeated using purified PMVs, which also selectively bind to GFP positive cells, in agreement with the results shown here. To confirm this finding, flow cytometry analysis was conducted on the co-cultured cells. The significant difference in green channel fluorescence signal was used to distinguish GFP positive and control cells on a cell-by-cell basis (FIG. 21C top). Only the GFP positive cells had a detectable increase in the mRFP fluorescence channel as a result of PMV binding (FIG. 21C bottom). The mean fluorescence increase in the mRFP channel for the two cell populations was calculated (FIG. 21D). The results indicated that the PMVs have a selectivity for GFP positive cells of approximately 50:1, which is comparable to the selectivity that chemically conjugated synthetic liposome particles can achieve for their target cells. Collectively, these results demonstrate that GFPnb-PMVs bind selectively to cells expressing GFP tagged transmembrane proteins.

Separate populations of CHO cells were transiently transfected with plasmids encoding recombinant proteins, one of which displays an extracellular GFP domain (FIG. 21E, top), while the other displays an intracellular GFP domain (FIG. 21E, bottom).

GPMVs were harvested from each population of transfected cells. Then the GPMVs were incubated with GFPnb PMVs. Line plots of the fluorescence intensity of the GPMV membranes showed that GFPnb PMVs bound to GPMVs displaying extracellular GFP. In contrast, no detectable binding was observed between GFPnb PMVs and GPMVs displaying intracellular GFP, indicating that a GFP domain expressed on the inner leaflet of the plasma membrane remains inaccessible on the surfaces of GPMVs. These results demonstrate that the process of harvesting GPMVs preserves the orientation of these model transmembrane proteins.

### Example 6—Evaluation of GPMVs In Vivo

A mouse breast tumor xenograft model is used to evaluate and optimize GPMVs for specific and efficient delivery of chemotherapeutics. Experiments are preformed to measure the circulation half-life of GPMVs and delivery of doxorubicin to tumors. The GPMVs are then further optimized in terms of drug loading and expression of connexins and targeting ligands. In addition, an in vivo study of the ability of GPMVs to drive breast tumor regression and prevent metastasis will be performed in comparison to clinical standards.

Measuring and Optimizing the Circulation Half-Life of GPMVs.

A key advantage of particle-based drug delivery is increased circulation time in comparison to direct delivery of drugs into the blood stream. Because GPMVs are derived from the plasma membrane of donor cells, they may have the potential for extended circulation, as demonstrated for other cell-derived vesicles (Thery et al., 2009) and materials (Parodi et al., 2013). To determine the circulation half-life for doxorubicin delivered to adult Sprague-Dawley rats, a single dose of 5 mg/kg is delivered using the following methods (3 rats per group): (i) free doxorubicin, (ii) liposomal doxorubicin (Doxil), (iii) doxorubicin-loaded GPMVs. Serial blood samples are collected at 0, 4, 8, 24, 48 hours after injection via the tail vein.

Measuring and Optimizing the Biodistribution of GPMVs.

A key therapeutic parameter is the percentage of the drug cargo loaded within GPMVs that reaches tumor cells. An orthotopic tumor model with the MDA 231 human breast cancer cell line injected into nu/nu 6-week old female mice (2×106 cells in the left #4 mammary gland) will be used. Treatment begins when tumors reach a volume of 500 mm³. The study examines the following groups (N=10 mice per group): (i) saline, (ii) free doxorubicin, (iii) liposomal doxorubicin (Doxil), (iv) doxorubicin encapsulated in GPMVs. Once tumors have been established a single dose of drug carrier particles encapsulating a total dose of 5 mg/kg doxorubicin is injected systemically via the tail vein. 72 hours after treatment, the animals are sacrificed. 5 mice per treatment group are analyzed for biodistribution of doxorubicin among blood, tumor, heart, kidney, liver, lung, skin, and spleen. For the remaining 5 mice per treatment group, tumors are analyzed by (i) immunohistochemical staining for gap junctions (connexin), apoptosis (cleaved caspase 3), and cell proliferation (Ki-67), and (ii) flow cytometric analysis and confocal imaging of doxobrubicin internalization by re-suspended tumor cells. Notably, staining experiments will reveal the degree of drug penetration and apoptosis in tumors, which is expected to correlate with the extent of gap junction expression. Analysis is performed in triplicate. Collectively these studies determine the extent to which GPMVs (i) localize drugs to tumors and (ii) reinforce gap junction networks.

Assessing the Potential of GPMVs to Drive Breast Tumor Regression and Suppress Metastasis.

To measure breast tumor regression and metastasis in vivo, two orthotopic tumor models with the MDA 231 human breast cancer cell line injected into nu/nu female mice and the 4T1.2 highly metastatic mouse mammary tumor cell line injected into syngeneic Balb/c mice are used. Treatment begins when tumors reach 500 mm³. The study examines the following groups (N=10 mice per group): (i) saline, (ii) free doxorubicin, (iii) liposomal doxorubicin (Doxil), (iv) doxorubicin encapsulated in GPMVs. For each group, a total of 15 mg/kg of doxorubicin are administered in three weekly doses of 5 mg/kg. Tumor volume is measured three times weekly by palpating tumors. Animals are sacrificed approximately one week after the final treatment or when tumor volumes exceed 750 mm3. Following sacrifice, metastases to the lung are visualized using India ink staining for the 4T1.2 group. Drug content and immunohistochemical staining is performed on all tumors. These studies reveal the extent to which delivering doxorubicin using GPMVs enhances tumor regression, suppresses metastasis, and restores the continuity of the gap junction network within breast tumors.

### Example 7—GPMVs for Cystic Fibrosis Treatment

Engineered GPMVs derived from the patient's own cells can be used for the development of a new treatment based on the direct delivery of wild-type (wt) CFTR transmembrane protein to the lungs. This strategy has not been previously pursued due to the technical difficulty associated with the incorporation of functional, properly-oriented CFTR transmembrane proteins in therapeutic particles. To overcome this challenge, pre-programmed GPMVs are engineered presenting a high concentration of functional wild-type CFTR in their lipid membranes. These GPMVs are extracted directly from the plasma membrane of healthy donor cells, through a process called cellular blebbing. The harvested GPMVs are then formulated as an aerosol for inhalation and delivered directly to the lung cells. The GPMVs will integrate into the membrane of defective lung epithelial cells by fusing with the plasma membrane or endosomes, thereby incorporating functional protein into the cells and ultimately normalizing their phenotype.

To maximize efficiency of CFTR delivery, in vitro experiments test: extraction of GPMVs from donor cells expressing wildtype CTFR and a fusogenic peptide and the proper insertion/integration of the CFTR protein into cell membrane using the developed GPMVs. To enhance the probability of fusion between the GPMVs and the recipient cells, a fusogenic peptide will be added to the GPMVs. Calu3 lung cells, naturally expressing high concentration of wildtype CFTR and engineered to express the fusogenic peptide Tat, will be used as the model donor cells. Tat has been previously used to increase transfection efficiency in lung cells in vitro (Renigunta et al., 2006) as well as pulmonary absorption of protein (Patel et al., 2009). CuFi-1 cells presenting ΔF508 mutation will be used as CF cellular model (Zabner et al., 2003). To extract CFTR-GPMVs Calu-3 cells will be grown to 90% confluency, and then they will be induced to bleb according to established protocol in Example 1. GPMVs will be collected and purified by centrifugation and extruded through a 1 μm membrane. The size and surface charge of the collected GPMVs will be measured by dynamic light scattering (DLS) and zeta potential, respectively. CFTR expression within the GPMVs will be assessed by confocal microscopy employing a fluorescently tagged anti-CFTR antibody. The concentration of GPMVs will be assessed using a hemocytometer and standardized to the protein content of samples, using a Bradford assay (Bio-Rad). Fluorescently-labeled, harvested GPMVs, will be added to CuFi-1 cells, presenting ΔF508 mutation. Cells will be imaged with a confocal microscope equipped with a chamber for live cell imaging (Zeis Axio Observer Z1). The interaction of the labeled GPMVs with the CuFi-1 cells will be analyzed to verify the delivery of CFTR. Biological functionality of the delivered CFTR protein to CuFi-1 cells will be determined using a whole-cell patch clamp assay (v10.2 Molecular Device) (Boinot et al., 2014). Chloride flow through CFTR will be measured in CuFi-1 cells by means of an electrode, upon application of the channel activator forskolin.

The vesicles are then aerosolized for pulmonary drug delivery (Smyth, 2003; El-Sherbiny et al., 2010) and their efficacy will be assessed in vitro and in vivo. The formulation will be optimized and tested in vitro according to the United States Pharmacopeia (USP) guidance. The in vivo efficacy will be tested in a mouse cystic fibrosis mouse model B6.129S6-Cftrtm1Kth/J, available from Jackson Laboratories. To develop the formulation and test it in vitro the GPMVs will be re-suspended in an isotonic buffer. Additional excipients for stability, sterility, and aerosol performance may be used to optimize the formulation. The formulation will be aerosolized using a conventional vibrating mesh nebulizer. The overall aerosol performance of the formulation will be assessed for delivery rate and total drug substance delivered according to the USP guidelines. The aerosolization and deposition properties of the formulation will be tested with a next generation pharmaceutical impactor (NGI, i.e. in vitro lung model). Biological activity and stability of the nebulized GPMVs will be tested by aerosol deposition directly onto cell monolayers. Using the TRANSWELL™ cell culture system, CuFi-1 cells will be placed within the in vitro lung model and exposed to the aerosol treatment. Patch clamp assay and confocal analysis will be performed on the cells. Untreated cells and cells treated with non-nebulized GPMVs will be used as negative and positive control, respectively. By leveraging GPMVs for CFTR protein delivery, this work is a fundamental departure from the conventional strategies of CFTR treatment and represents a breakthrough in the treatment of this deadly disease with enormous benefits for patients.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Al-Dosari, M. S. and X. Gao, AAPS J, 11(4): 671-81, 2009.
Al-Ghamdi, S. S., Drug Metab Lett 2, 47-50, 2008.
Allen, M. J., et al., The Journal of biological chemistry 286, 22139-22146, 2011.
Andrade-Rozental, A. F. et al., Brain Res Brain Res Rev, 32 (1), 308-15, 2000.
Avanzo, J. L. et al., Carcinogenesis, 25, 1973, 2004.
Barenholz, Y., Journal of Controlled Release 160, 117-134, 2012.
Bodenstine, T. M. et al., FEBS letters, 586, 27, 2012.
Boinot, C., et al., J Pharmacol Exp Ther, 350(3): 624-34, 2014.
Chen, S. S. et al., Cell Growth & Differentiation, 6, 681, 1995.
Connors, B. W., Epilepsy Curr 12, 66-68, 2012.
Eghbali, B. et al., PNAS, 88, 10701, 1991.
El-Sherbiny, I. M. and H. D. Smyth, Int J Pharm, 395(1-2): 132-41, 2010.
Flotho, A., et al., In Ubiquitin Family Modifiers and the Proteasome: Reviews and Protocols (Dohmen, R. J., and Scheffner, M., Eds.), pp 93-110, 2012.
Fong, J. T., et al., Autophagy 8, 794-811, 2012.
Fujimoto, W. Y. et al., PNAS, 68 (7), 1516-9, 1971.
Garcia-Rodriguez, L. et al., Mol Cancer Ther, 10 (3), 505-17, 2011.
Gibbons and Smyth, Controlled Pulmonary Delivery: 223-236, 2011.
Gottesman, M. M., et al., Nat Rev Cancer 2, 48-58, 2002.
Griesenbach, U. and E. W. Alton, Curr Pharm Des, 18(5): 642-62, 2008.
Huang, R. P. et al., Int J Cancer, 92 (1), 130-8, 2001.
Horowitz, A. T., et al., Biochimica Et Biophysica Acta 1109, 203-209, 1992.
Kaoud, T. S. et al., ACS Chemical Biology, 6, 658, 2011.
Kim, D. Y. et al., J Biol Chem, 274 (9), 5581-7, 1999.
Kaneda, M. et al., Biomaterials, 30 (23-24), 3971-7, 2009.
Ishida, T. et al., International Journal of Pharmaceutics, 354 (1-2), 56-62, 2008.
International Publication No. WO89/008846.
Chen, L. et al., Journal of the American Chemical Society, 132 (10), 3628-35, 2010.
Kirpotin, D. et al., Biochemistry, 36, 66, 1997.
Lauf et al., PNAS, 99:10446-51, 2002.
Lane et al., Scientific Reports, 5, 2015.
Lauf, U., et al., Proceedings of the National Academy of Sciences of the United States of America 99, 10446-10451, 2002.
Li, Z. et al., Clinical & Experimental Metastasis, 25, 893, 2008.
Mamot, C. et al., Cancer Research, 63, 3154, 2003.
Mehta, P. P. et al., Cell, 44, 187, 1986.
Mesnil, M. Biology of the Cell, 94, 493, 2002.
Mesnil, M. et al., PNAS, 93 (5), 1831-5, 1996.
Kam, Y. et al., Biochim Biophys Acta, 1372 (2), 384-8, 1998.
Muller, D. J., et al., Embo Journal 21, 3598-3607, 2002.
Naus, C. C. and Laird, D. W. Nature Reviews. Cancer, 10, 435, 2010.
Ng, D. P., et al., Biochim Biophys Acta, 1818(4): 1115-22, 2012.
Parodi, A. et al., Nature nanotechnology, 8, 61, 2013.
Patel, L. N., et al., Mol Pharm, 6(2): 492-503, 2009.
Reaume et al., Science, 267:1831-1834, 1995.
Renigunta, A., et al., Bioconjug Chem, 17(2): 327-34, 2006.
Riordan, J. R., Annu Rev Biochem, 2008. 77: 701-26, 2008.
Roovers, R. C. et al., Cancer Immunology, Immunotherapy, 56, 303, 2007.
Sakhtianchi, R., et al., Colloid and Interface Science. 201, 18-29, 2013.
Schmalhofer, O. et al., Cancer Retastasis Reviews, 28, 151, 2009.
Sezgin, E., et al., Nature Protocols 7, 1042-1051, 2012.
Shao, Q. et al., Cancer Research, 65, 2705, 2005.
Smyth, H. D., Adv Drug Deliv Rev, 55(7): 807-28, 2003.
Thery, C. et al., Nature Reviews. Immunology, 9, 581, 2009.
Thimm, J., et al. Journal of Biological Chemistry 280, 10646-10654, 2005.
U.S. Pat. No. 5,008,050.
U. S. Patent Application Nos. 2013/0273544 and 2015/0104801.
Unwin, P. N. T., and Zampighi, G., Nature 283, 545-549. 1980.
Yamasaki et al., Carcinogenesis, 17:1199-1213, 1996.
Yamasaki, H. et al. C R Acad Sci III, 322 (2-3), 151-9, 1999.
Ye, Z. C. et al., Glia, 57, 258, 2009.
Zabner, J., et al., Am J Physiol Lung Cell Mol Physiol, 284(5): L844-54, 2003.

1. A vesicle comprising a phospholipid membrane wherein the phospholipid membrane comprises a recombinant transmembrane protein and a fusogenic peptide, wherein said protein and peptide are embedded in said membrane.

2. The vesicle of claim 1, wherein the recombinant transmembrane protein is a transporter, receptor, channel, cell adhesion protein, or enzyme.

3. The vesicle of claim 1, wherein the recombinant transmembrane protein is a connexin, cystic fibrosis transmembrane conductance regulator (CFTR), thyrotropin receptor, myelin protein zero, melacortin 4, myelin proteolipid protein, low-density lipoprotein receptor, or ABC transporter.

4. The vesicle of claim 1, wherein the recombinant transmembrane protein is Connexin 43 or CFTR.

5. The vesicle of claim 1, wherein about 100,000 to about 500,000 recombinant transmembrane proteins are embedded in the phospholipid membrane.

6. The vesicle of claim 1, wherein the vesicle further comprises a small molecule, peptide, nucleic acid molecule, or RNAi.

7. The vesicle of claim 1, wherein the vesicle further comprises a chemotherapeutic drug.

8. The vesicle of claim 7, wherein the chemotherapeutic drug is doxorubicin, etoposide, paclitaxel, or gemcitabine.

9. The vesicle of claim 1, wherein the vesicle further comprises a targeting molecule.

10. The vesicle of claim 9, wherein the targeting molecule comprises an antibody or fragment thereof, a polypeptide, a dendrimer, an aptamer, an oligomer or a small molecule.

11. The vesicle of claim 9, wherein the targeting molecule has an affinity for a receptor expressed in cancer cells.

12. The vesicle of claim 9, wherein the targeting molecule binds to human epidermal growth factor receptor (EGFR), vascular endothelial growth factor receptor, folic acid receptor, melanocyte stimulating hormone receptor, integrin avb3, integrin avb5, transferrin receptor, interleukin receptors, lectins, insulin-like growth factor receptor, hepatocyte growth factor receptor or basic fibroblast growth factor receptor.

13. The vesicle of claim 10, wherein the antibody fragment is an EGFR single-domain antibody fragment.

14. The vesicle of claim 1, wherein the vesicle has a diameter of less than about 20 μm.

15. (canceled)

16. The vesicle of claim 1, wherein the fusogenic peptide comprises trans-activating transcriptional activator (TAT) or TAT-HA2.

17. A method of producing the vesicle of claim 1 comprising:
(a) providing a donor cell, wherein the donor cell is genetically engineered to express the recombinant transmembrane protein;
(b) contacting the donor cell with a blebbing buffer, under conditions effective to induce donor cell blebbing; and
(c) harvesting the vesicle from the blebbing buffer.

18.-26. (canceled)

27. A method of treating a disease or disorder in a subject comprising administering a therapeutically effective amount of the vesicle of claim 1.

28. The method of claim 27, wherein the disease is cancer.

29. (canceled)

30. The method of claim 27, wherein the disease is cystic fibrosis.

31.-32. (canceled)

33. The method of claim 27, wherein the disease is a skin disease.

34.-36. (canceled)


SEQUENCE LISTING (2 sequences)

SEQ ID NO: 1 - protein, 24 aa, Artificial sequence
  Synthetic peptide
GLFEAIAGFIENGWEGMIDGGGYC

SEQ ID NO: 2 - protein, 11 aa, Artificial sequence
  Synthetic peptide
YGRKKRRQRRR